(19) United States
(12) Patent Application Publication
(10) Pub. No.: US 2021/0151312 A1
(43) Pub. Date: May 20, 2021

(54) METHODS FOR LASER DESORPTION IONIZATION MASS SPECTROSCOPY BASED IMAGING OF NEUROTRANSMITTERS AND METABOLITES USING NANOPARTICLES

(71) Applicant: The University of Scranton, Scranton, PA (US)

(72) Inventors: Katherine Stumpo, Scranton, PA (US); Nolan McLaughlin, Dunmore, PA (US); Tyler Bielinski, Dunmore, PA (US); Kristine Glunde, Baltimore, MD (US); Catlin Tressler, Middle River, MD (US)

(21) Appl. No.: 17/097,189

(22) Filed: Nov. 13, 2020

Related U.S. Application Data
(60) Provisional application No. 62/935,882, filed on Nov. 15, 2019.

(51) Int. Cl. H01J 49/16; H01J 49/04; H01J 49/00

(52) U.S. Cl. CPC H01J 49/164; H01J 49/0409; H01J 49/0004; H01J 49/0036; H01J 49/0445

(57) ABSTRACT

The present disclosure provides methods of imaging neurotransmitters and metabolites present in a biological sample comprising: pneumatically spraying or having sprayed the biological sample with a nanoparticle, introducing the sample to a laser desorption ionization mass spectrometer to collect mass spectral data and identifying the neurotransmitters or metabolites in the sample based on the mass spectral data.

### CROSS-REFERENCE TO RELATED APPLICATIONS

This application claims priority to U.S. provisional Application No. 62/935,882 filed on Nov. 15, 2019 which is incorporated herein by reference in its entirety.

### FIELD

The present disclosure is directed in part to methods of imaging neurotransmitters and metabolites in a biological sample. Specifically, a biological sample is pneumatically sprayed with a nanoparticle solution and the sample is then introduced into a laser desorption ionization mass spectrometer to collect mass spectral data. The mass spectral data is collected and used to identify neurotransmitters or metabolites present in the biological sample.

### BACKGROUND

Neurotransmitters are extremely important in biological systems. Specifically, dopamine, serotonin, octopamine, norepinephrine, epinephrine, acetylcholine, gamma amino butyric acid (GABA) and glutamate have been implicated in behavioral, developmental and emotional regulation. To best understand the function of these neurotransmitters it is important understand their location and concentration in the body. Matrix-assisted laser desorption ionization (MALDI) is usually a three step process. First a matrix solution is mixed with the analyte if it is available in a purified form. Suitable matrix solutions are known in the art and are often acidic, to serve as a proton donor to encourage ionization of the analyte although basic matrices have also been used (often depending on the mode of ionization, for positive or negative ions). Matrices typically have a strong optical absorption in either the UV or visible range so that they rapidly and efficiently absorb the laser irradiation. This is commonly associated with the presence of several conjugated double bonds are also commonly functionalized with polar groups, allowing their use in aqueous solutions.

A matrix solution that is a mixture of water and organic solvent allows both hydrophobic and hydrophilic molecules to dissolve into the solution. This solution is spotted onto a MALDI plate (usually a metal target plate designed for this purpose). The solvents will vaporize, leaving a crystallized matrix with analyte molecules embedded into the crystal structure. The plate is then introduced into the mass spectrometer. In the next step a pulsed laser irradiates the sample, causing ablation and desorption of the sample and matrix material. Finally, the analyte molecules are ionized by being protonated or deprotonated in the plume of ablated material and then they can be accelerated into the mass spectrometer for detection.

MALDI has been used to ionize small molecules but generally these methods suffer from poor resolution and high background chemical noise due to organic matrices. Neurotransmitters in particular, have been difficult to detect in situ using traditional MALDI techniques due to their low abundance and low molecular mass resulting in high chemical noise with most traditional matrices. Furthermore previous studies have required chemical derivation of the neurotransmitters to facilitate ionization and analysis and the additional steps and time required for derivation affect the ability to achieve high throughput examination of samples and may increase the chances of spatial movement or delocalization of the neurotransmitter in an organ, tissue or cell.

Accordingly, there is a need for methods which provide reduced time and cost alternatives to existing methods. Specifically, there is a need for methods which reduce or eliminate delocalization, increase reproducibility, and provide long-term stability of readily prepared tissue samples while increasing the ability to ionize neurotransmitters and metabolites and identify them in situ.

### SUMMARY

The present invention provides a method of imaging neurotransmitters in a biological sample comprising: pneumatically spraying or having sprayed a biological sample with a nanoparticle; introducing the sample into a laser desorption ionization mass spectrometer to collect mass spectral data and identifying the neurotransmitters in the sample based on the mass spectral data.

The present invention also provides methods wherein a neurotransmitter is a monoamine such as dopamine, octopamine, norepinephrine, epinephrine, serotonin, and histamine, or the neurotransmitter is an amino acid such as glutamate, gamma-aminobutyric acid (GABA), glycine, and tyramine.

The present invention also provides methods wherein the neurotransmitter is acetylcholine, adenosine or nitric oxide.

The present invention also provides methods wherein the neurotransmitter is present in the biological sample at physiological concentration.

The present invention also provides methods wherein said biological sample is an organ, tissue, or cell such as brain tissue, spinal tissue, or peripheral nerve tissue. The present invention also includes methods wherein the biological sample is present in neural tissue.

The present invention also provides methods wherein the pneumatic spraying is done with a high volume, low pressure device.

The present invention also provides methods wherein the spraying is done using a hand spraying device such as an airbrush.

The present invention also provides methods wherein the spraying is done at a temperature of from about 22° C. to about 95° C. and at a velocity of about 1000 mm/minute to about 2000 mm/minute.

The present invention also provides methods wherein the nanoparticle is gold, silver, platinum, or silica.

The present invention also provides methods wherein a nanoparticle is coated with gold, silver, or platinum.

The present invention also provides methods wherein the nanoparticle is solid, hollow, a pitted solid, or has at least one open channel therein or is a solid with an exterior coating.

The present invention also provides methods wherein the nanoparticle is silica coated with gold.

The present invention also provides methods wherein the nanoparticle is substantially in the shape of a sphere, wire, rod, pyramid, double pyramid, diamond, cube, or star.

The present invention also provides methods wherein a negatively charged surface ligand is adsorbed on the surface of the nanoparticle. The present invention also provides methods wherein the negatively charged ligand is a ligand such as a carboxylic acid functionality.

The present invention also provides methods wherein the negatively charged ligand is a carboxylic acid functionality such as citrate.

The present invention also provides methods wherein a positively charged surface ligand is adsorbed on the surface of the nanoparticle. The present invention also provides methods wherein the positively charged surface ligand is a ligand such as a quaternary amine.

The present invention also provides methods wherein a neutrally charged surface ligand is adsorbed on the surface of the nanoparticle. The present invention also provides methods wherein the neutrally charged surface ligand is a ligand such as tannic acid, dextrin, and dextrans.

The present invention also provides methods wherein a fluorescent ligand is adsorbed on the surface of the nanoparticle.

The present invention also provides methods wherein the fluorescent ligand is covalently bound to the surface of said nanoparticle.

The present invention also provides methods wherein said nanoparticle ranges from a about 1 nm to about 50 nm in their longest dimension.

The present invention also provides methods wherein said nanoparticle is coated with both a metal and a fluorescent ligand.

The present invention also provides methods wherein mass spectral data are obtained for more than one neurotransmitter in said biological sample.

The present invention also provides methods for imaging metabolites present in a biological sample comprising: pneumatically spraying or having sprayed said biological sample with a nanoparticle; introducing said sample to a laser desorption ionization mass spectrometer to collect mass spectral data; identifying the metabolite in the sample based on the mass spectral data.

The present invention also provides methods wherein the metabolite is selected from the group consisting of glucose, pyruvate, NAD, NADH, ATP, ADP, FAD, and FADH.

The present invention also provides a mass spectrometer sample prepared by pneumatically spraying or having sprayed a biological sample with a nanoparticle.

The present invention also provides a biological sample prepared by pneumatically spraying or having sprayed a biological sample with a nanoparticle for analysis in a mass spectrometer.

### BRIEF DESCRIPTION OF THE DRAWINGS

The patent application file contains at least one drawing executed in color. Copies of this patent or patent application publication with color drawing(s) will be provided by the Office upon request and payment of the necessary fee.

FIG. 1 shows positive ion LDI mass spectrum of homogenized crayfish brain using 2 nm AuNPs with an intensity of 680 mV (Panel A), SA with an intensity of 1382 mV (Panel B), and DHB with intensity of 144 mV (Panel C). All detected NTs are labeled as well as the Au⁺ ion. Data was collected on the target plate as a proof-of-concept for NT ionization and not via pneumatic spraying.

FIG. 2 shows positive ion LDI mass spectrum of human serum using 2 nm AuNPs. All detected NTs are labeled as well as the Au⁺ ion. Data was collected on the target plate as a proof-of-concept for NT ionization and not via pneumatic spraying.

FIG. 3 shows MSI of coronal rabbit brain tissue section at 20 μm lateral spatial resolution. Panel A shows the optical image with annotations for white matter (WM), gray matter (GM), and ventricles (V). Panel B shows the DA/OT image at m/z 154.02. Panel C shows the NE image at m/z 169.97. Panel D shows the GABA/choline image at m/z 104.04.

FIG. 4 shows positive ion LDI mass spectra of 10 μm sliced rabbit brain tissue section, normalized to TIC. Panel A shows the CHCA spectrum with an intensity of 825 a.u. and Panel B shows the 2 nm AuNP spectrum with an intensity of 6.55 a.u. All detected NTs are labeled. Improvement in NT detection was achieved with pneumatic spraying of NPs vs. organic acid matrix.

FIG. 5 shows MSI of axial zebrafish embryo tissue section at 20 μm lateral spatial resolution. Panels A and D show the GABA/choline images at m/z 104.12. Panels B and E show the 5-HT images at m/z 177.09. Panels C and F show the EP images at m/z 181.05. The repeated trials over multiple days is an advancement from organic acid matrix methods.

FIG. 6 shows MSI of a sagittal zebrafish tissue section at 5 μm lateral spatial resolution. Panel A shows the optical image with eye (E), forebrain (FB), midbrain (MB), and hindbrain (HB) indicated. Panel B shows the GABA+Na⁺ image at m/z 126.01. Panel C shows the EP image at m/z 184.32. Panel D shows the histidine image at m/z 156.07. Panel E shows the ACh image at m/z 146.10. Panel F shows the GLU image at m/z 147.09. Panel G shows the DA/OT image at m/z 154.09. Panel H shows the NE image at m/z 170.11. Panel I shows the 5-HT image at m/z 177.22.

FIG. 7 shows MSI of neuroblastoma cells at 5 μm lateral spatial resolution. Panel A shows a confocal optical image depicting the largest grouping of cells. Panel B shows the [GLU+K]⁺ adduct image at m/z 185.02. Panel C shows the GABA/choline image at m/z 104.03. Panel D shows the glucose image at m/z 181.00.

FIG. 8 shows LDI mass spectra for acetylcholine using 2 nm AuNPs and 5 nm AuNPs, and with analyte alone. Data was collected on the target plate as a proof-of-concept for NT ionization and not via pneumatic spraying.

FIG. 9 shows LDI mass spectra for dopamine using 2 nm AuNPs and 5 nm AuNPs. Data was collected on the target plate as a proof-of-concept for NT ionization and not via pneumatic spraying.

FIG. 10 shows LDI mass spectra for epinephrine using 2 nm AuNPs and 5 nm AuNPs. Data was collected on the target plate as a proof-of-concept for NT ionization and not via pneumatic spraying.

FIG. 11 shows LDI mass spectra for 4-amino butyric acid using 2 nm AuNPs and nm AuNPs. Data was collected on the target plate as a proof-of-concept for NT ionization and not via pneumatic spraying.

FIG. 12 shows LDI mass spectra for glutamate using 2 nm AuNPs and 5 nm AuNPs. Data was collected on the target plate as a proof-of-concept for NT ionization and not via pneumatic spraying.

FIG. 13 shows LDI mass spectra for serotonin using 2 nm AuNPs and 5 nm AuNPs. Data was collected on the target plate as a proof-of-concept for NT ionization and not via pneumatic spraying.

FIG. 14 shows LDI mass spectra for norepinephrine using 2 nm AuNPs and 5 nm AuNPs. Data was collected on the target plate as a proof-of-concept for NT ionization and not via pneumatic spraying.

FIG. 15 shows representative LDI mass spectrum for acetylcholine using organic matrix.

FIG. 16 shows LDI mass spectra for human serum with spiked NTs using CHCA, DHB, and SA.

FIG. 17 shows images of rabbit brain for protonated and salt adducts. The ability to detect more than one ion that corresponds to a NT allows for more complete verification of said NT being present. NPs allow for multiple ions to be detected.

FIG. 18 shows LDI MS/MS of m/z 184 ionized on zebrafish tissue section sprayed with AuNPs.

FIG. 19 shows images of rabbit brain using CHCA. Ion intensity outside the tissue margins (bold white line) is a problem with analyte delocalization with organic matrices.

FIG. 20 shows LDI average mass spectra of zebrafish embryos tissue collected on day 1 and day 2.

FIG. 21 shows LDI MS/MS of m/z 126 ionized on zebrafish tissue section sprayed with AuNPs.

FIG. 22 shows LDI MS/MS of m/z 146 ionized on zebrafish tissue section sprayed with AuNPs.

FIG. 23 shows LDI MS/MS of m/z 147 ionized on zebrafish tissue section sprayed with AuNPs.

FIG. 24 shows LDI MS/MS of m/z 154 ionized on zebrafish tissue section sprayed with AuNPs.

FIG. 25 shows LDI MS/MS of m/z 156 ionized on zebrafish tissue section sprayed with AuNPs.

FIG. 26 shows LDI MS/MS of m/z 170 ionized on zebrafish tissue section sprayed with AuNPs.

FIG. 27 shows LDI MS/MS of m/z 177 ionized on zebrafish tissue section sprayed with AuNPs.

FIG. 28 shows LDI MS/MS of m/z 181 ionized on zebrafish tissue section sprayed with AuNPs.

FIG. 29 shows skyline spectrum for zebrafish embryo (20 μm lateral spatial resolution).

FIG. 30 shows skyline spectrum for zebrafish embryo (5 μm lateral spatial resolution).

FIG. 31 shows LDI mass spectra of NE using 2 nm, 5 nm, HCCA, and DHB as matrices. Asterisks represent matching EI spectra fragmentation and purple asterisks highlight different fragment ions not found in a 2 nm AuNP spectrum or different abundances with respect to the base peak. Orange asterisks indicate the presence of [M+OH]⁺.

FIG. 32 shows LDI mass spectra of 5-HT using 2 nm, 5 nm, HCCA, and DHB as matrices. Orange asterisks indicate the presence of a product indicative of a hydroxyl initiated reaction, namely [M+OH]⁺.

FIG. 33 shows LDI mass spectra of zebrafish embryos at 24-36 hours post fertilization using 5 nm AuNPs. Data was collected without the use of a pneumatic sprayer and AuNPs were just dried on top of the tissue.

FIG. 34 shows MSI of 10 μm coronal zebrafish whole body tissue section imaged at 5 μm lateral spatial resolution. [Dopamine/Octopamine+H]⁺ is shown with varying linear flow rates and spray temperatures with 1500 mm/min spraying velocity at 30° C. (Panel A), 1900 mm/min at 30° C. (Panel B), 1500 mm/min spraying velocity at 45° C. (Panel C), 1500 mm/min at 60° C. (Panel D), and 1500 mm/min at 75° C. (Panel E). The flexible conditions used that still result in data is in contrast to organic acid matrices, which do not have such flexibility.

FIG. 35 shows MSI of 10 μm coronal zebrafish whole body tissue section imaged at 5 μm lateral spatial resolution. GABA/Choline+H is shown with varying organic:aqueous with 80/20 MeOH/H₂O ratio (Panel A), 100% H₂O at 1900 mm/min spraying velocity (Panel B), and 100% H₂O at 1500 mm/min spraying velocity (Panel C).

FIG. 36 shows MSI of 10 μm coronal zebrafish whole body tissue section imaged at 5 μm lateral spatial resolution. [Dopamine/Octopamine+H] is shown with one spray pass (Panel A), five spraying passes (Panel B), and ten spraying passes (Panel C).

FIG. 37 shows comparison of the ionization intensities across multiple sample preparation methods in arbitrary unites (a.u.) for protonated histamine (m/z=112). Having a higher intensity range is indicative of better ionization. These comparisons were for AuNPs using different spray conditions.

FIG. 38 shows scores and Scree Plots of Principal Component Analysis (PCA) generated using data that compares different preparation methods using 5 nm AuNPs. Orange scores are an 80:20 ratio of methanol to water with normal velocity. Light blue scores are 100% water at high velocity. Purple scores are 100% water with normal velocity. The divergence of orange/blue from purple indicates distinct differences in ionization capabilities of the conditions analyzed.

FIG. 39 shows comparison between 5 nm AuNPs with varied preparation conditions analyzing ionization in arbitrary units (a.u.) of the neurotransmitters acetylcholine and GABA/choline. Having a higher intensity range is indicative of better ionization. These comparisons were for AuNPs using different adsorbed species on the 5 nm AuNP.

FIG. 40 shows images for GABA/choline from 10 mm zebrafish tissue sections (at 20 mm lateral spatial resolution) ionized using pneumatically sprayed 5 nm AuNPs with MeOH:H₂O ratios of 80:20 (Panel A), 0:100 (Panel B), and 0:100 (Panel C).

FIG. 41 shows Principal Component Analysis (PCA) on multiple biological samples. Panels C and F show delocalization in the images and PCs that show variability which do not correspond to anatomical features. Panels A, B, D, and E show anatomical features of the zebrafish qualitatively visualized in the PCs.

### DESCRIPTION OF EMBODIMENTS

The terminology used herein is for the purpose of describing particular embodiments only and is not intended to be limiting.

The present disclosure provides methods of imaging neurotransmitters present in a biological sample by pneumatically spraying or having sprayed a biological sample with a nanoparticle, introducing the sample to a laser desorption ionization mass spectrometer to collect mass spectral data and identifying the neurotransmitters in the sample based on the mass spectral data.

In some embodiments a sample may be sprayed and stored or sent to another location for analysis. In other embodiments a sample is analyzed soon after spraying close or at the site of sample preparation.

A neurotransmitter is a chemical substance that is released at the end of a nerve fiber by the arrival of a nerve impulse and, by diffusing across the synapse or junction, causes the transfer of the impulse to another nerve fiber, a muscle fiber, or some other structure. A wide variety of neurotransmitters may be identified using the methods of the present invention.

In some embodiments the neurotransmitter is a monoamine. Monoamines include but are not limited to dopamine, octopamine, norepinephrine, epinephrine, serotonin, and histamine.

In other embodiments the neurotransmitter is an amino acid. Amino acid neurotransmitters include but are not limited to glutamate, gamma aminobutyric acid (GABA), glycine, and tyramine.

The present invention may also be used to identify other kinds of neurotransmitters that include but are not limited to acetylcholine, adenosine, and nitric oxide.

The present methods also offer the ability to detect neurotransmitters at physiological concentrations. The physiological concentration of a neurotransmitter in a biological sample may vary based on the specific neurotransmitter, the type of sample, developmental progress, and the organism used to obtain the sample. Accurate determination of the locations and concentrations of neurotransmitters or metabolites in biological samples offers important insights on where and how these neurotransmitters function and may suggest approaches to treat or cure a variety of diseases that involve these neurotransmitters or metabolites. The biological samples are obtained using a variety of methods known in the art and may be prepared in a variety of ways as discussed herein.

The biological samples used in the present methods may come from any animal species. Such animals include but are not limited to vertebrates including mammals such as humans. Animals typically used in studies of neurotransmitters include but are not limited to rats, mice, rabbits, crayfish, and zebrafish. Biological samples can also be obtained from xenografts, knockout, and transgenic organisms.

In certain embodiments samples obtained from cell lines, in particular vertebrate cell lines may be studied.

The present invention provides the ability to identify neurotransmitters in biological samples obtained from an organ, a tissue, or a cell. Organs include organs that are obtained from the integumentary, skeletal, muscular, nervous, endocrine, cardiovascular, lymphatic, respiratory, digestive, urinary, and reproductive systems. In certain embodiments organs are obtained from the nervous system such as the brain and spinal cord.

The present invention provides for the identification of neurotransmitters in a variety of tissues. In certain embodiments the biological sample is brain tissue spinal tissue or peripheral nerve tissue. In certain embodiments the biological sample is present in neural tissue. In certain embodiments the methods of the present invention are used for the identification of neurotransmitters present in brain tissue.

A pneumatic spray device may be used in the present invention to spray the nanoparticles onto a biological sample such that the nanoparticles are able to come into contact with neurotransmitters or metabolites. In certain embodiments a high volume low pressure device is used. A number of spraying devices are commercially available such as those available from HTX Technologies. These systems can provide an automated process for sample preparation and provide fine uniform and consistent matrix coating which is important for high-resolution imaging and the relative quantification of neurotransmitters. The liquid and propulsion gas temperature can be controlled to create a fine solution mist that is deposited in a precise and adjustable pattern over all or part of any sample containing and specific spray volume, pressure, and other characteristics (wet or dry) are easily adjustable to optimize preparation of a sample. In certain other embodiments the spraying may be done using a hand spraying device such as a handheld device or using airbrushes such as those available from Badger, Iwata, Master, Harder & Steenbeck or Paasche.

The mass spectrometer will commonly have a built-in chamber with a door for access and inserting the target plate (which may contain slides bearing the pneumatically sprayed tissue slices). For sources that operate under vacuum, the chamber will close, and a vacuum pump will reduce the pressure. Once the source is at a pressure compatible with the rest of the mass spectrometer, experimentation can begin.

Alternatively, an atmospheric pressure source would not evacuate the sample chamber and would instead have an opening to the mass spectrometry whereby ionized molecules would be drawn to enter the instrument because of the change in pressure (the instrument is still under vacuum).

The nanoparticles to be sprayed are in are a colloidal suspension (a homogeneous noncrystalline substance consisting of large molecules or ultramicroscopic particles of one substance dispersed through a second substance.

The volume of nanoparticle containing spray that is sprayed on a target will vary but in general the volume should be sufficient to cover the biological sample and will have an approximate spray density of 4×10¹² mg NP/mm². The spray velocity can be adjusted to provide efficient coverage of the biological sample while avoiding causing displacement of the analyte molecules and may range from about 1000 mm/min to about 2000 mm/min. The spraying can be done at a variety of temperatures ranging from about 22° C. to about 95° C. A variety of laser desorption ionization mass spectrometers (LDI-MS), often called matrix assisted laser desorption ionization mass spectrometry (MALDI-MS) may be used in the practice of the invention. Examples of suitable commercially available LDI-MS include but are not limited to spectrometers made by Bruker, Shimadzu, SciEx, ThermoFisher, Agilent and Waters. Alternatively, a custom built LDI-MS may be used.

The spectrometers typically use one of two standard UV lasers; frequency-tripled Nd:YAG at 355 nm or a nitrogen laser at 337 nm.

Matrix-assisted laser desorption/ionization combined with laser-induced postionization (MALDI-2) might also be used using the methods of the present invention.

The methods of the present invention can utilize nanoparticles made from a variety of materials.

In certain embodiments the nanoparticle is made of metal such as gold, silver or platinum. In certain embodiments the nanoparticle is made of gold. In certain other embodiments the nanoparticle is made of silica The nanoparticles can be solid, hollow, pitted solids or have at least one open channels through the particle. The nanoparticles of the present invention may be in a variety of shapes accordingly, nanoparticles that are substantially in shapes that include but are not limited to a sphere, a wire, a rod, a pyramid, a double pyramid, a diamond, a cube or a star may be used. In certain embodiments the nanoparticle will be in the shape of a sphere.

In some embodiments uncoated nanoparticles are used however in some embodiments it may be useful to add an outer coating to a nanoparticle based on a variety of considerations such as cost, the desire to have an active surface coating, physiological interactions, the density of the material the ability to do other chemistry or modifications to the particle and the ability to manufacture the nanoparticle. In some embodiments the nanoparticle may be solid with an exterior coating.

In some embodiments a nanoparticle such as a silica nanoparticle can be coated with a metal such as gold, silver or platinum. In certain embodiments the nanoparticle is silica covered with gold.

In certain embodiments of the invention a negatively charged surface ligand is absorbed on the surface of the nanoparticle. Negatively charged surface ligands include but are not limited to a carboxylic acid functionality. In certain embodiments the ligand is a carboxylic acid functionality. In certain embodiments the carboxylic acid functionality is citrate. In some embodiments a gold nanoparticle covered with citrate is used.

In certain other embodiments a positively charged surface ligand is absorbed on the surface of the nanoparticle. Positively charged ligands include but are not limited to quaternary amines. In certain embodiments the positively charged ligand is a quaternary amine.

In certain embodiments a neutral charged surface ligand is absorbed on the surface of a nanoparticle. Neutrally charged ligands include but are not limited to tannic acid, dextrin or dextrans.

In yet other embodiments a fluorescent ligand is absorbed on the surface of the nanoparticle. The florescent ligand allows the location of a nanoparticle to be determined by fluorescence. Biological tissue stained with fluorescent dyes will fluoresce and give information on cell type and cellular features. Once fluorescent staining is done, the tissue sample can no longer be used for any other purpose. Tissue slices that are used for mass spectrometry imaging can be used to collect mass spectral data and then stained after those experiments, but then cannot be used for additional imaging experiments.

In some embodiments the nanoparticles of the present invention include both a metal coating and a fluorescent ligand.

Combining gold nanoparticles with fluorescent tags may allow for MSI experiments and fluorescent imaging, without the need for the destruction of tissue using fluorescent stains. Adsorbed fluorescent ligands may or may not be covalently bound to the nanoparticle. If a covalently bound ligand is used a nanoparticle may be covalently bound to a fluorescent ligand using a thiol or amine linker. The fluorescent ligands may have fluorophore functional groups from the broad classes of fluorescein dyes such as but not limited to Alexa® dyes, coumarin dyes, DYT dyes, and rhodamine dyes.

The nanoparticles used in the present invention may range in size from a diameter of about 1 to about 50 nm nanometers in their longest dimension.

The methods of the present invention can be used also facilitate obtaining mass spectral data for more than one kind of neurotransmitter present in the same biological sample.

A metabolite is a substance formed in or necessary for metabolism, or as part of a metabolic pathway, within a healthy, diseased, or drug-administered organism/tissue. Metabolites can also be defined by function, including fuel, structure, signaling, catalysis, and defense. A variety of metabolites may be present in a biological sample.

The present invention also provides methods of imaging metabolites present in a biological sample by spraying or having sprayed a biological sample with a nanoparticle then introducing the sample to a laser desorption ionization mass spectrometer collecting the mass spectral data and identifying the metabolite based on the mass spectral data.

Metabolites which can be detected using the methods of the present invention include but are not limited to, glucose, pyruvate, NAD, NADH, ATP, ADP, FAD, and FADH.

In order that the subject matter disclosed herein may be more efficiently understood, examples are provided below. It should be understood that these examples are for illustrative purposes only and are not to be construed as limiting the claimed subject matter in any manner.

### EXAMPLES

### Example 1: Pneumatically Sprayed Gold Nanoparticles for Mass Spectrometry Imaging of Neurotransmitters

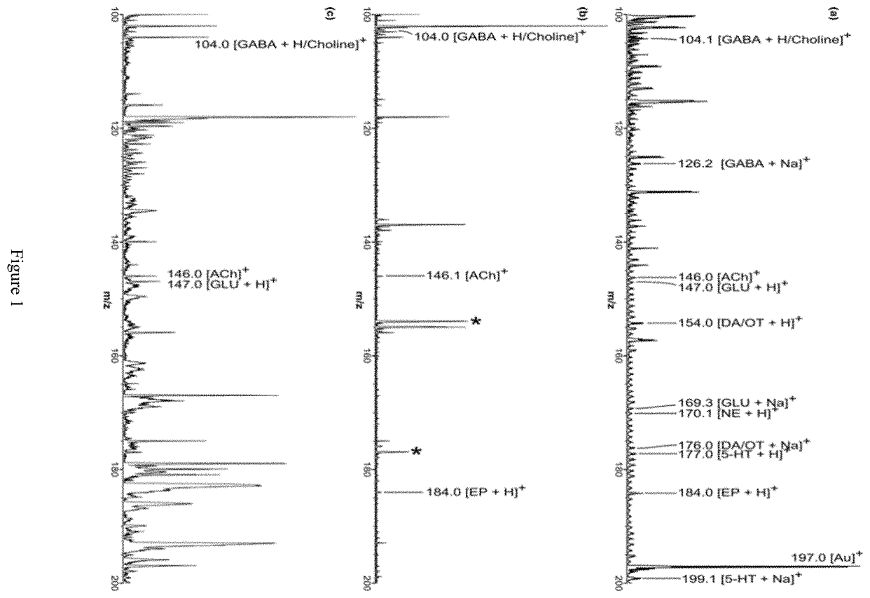

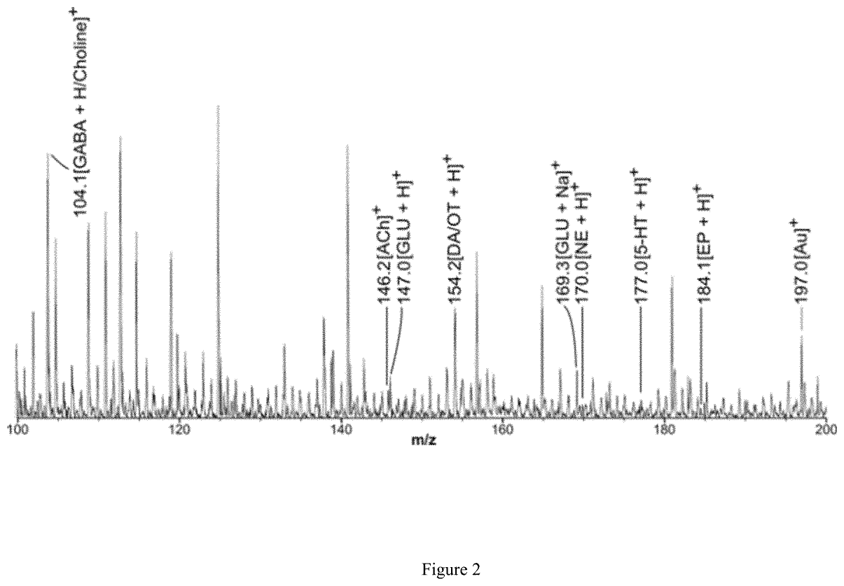

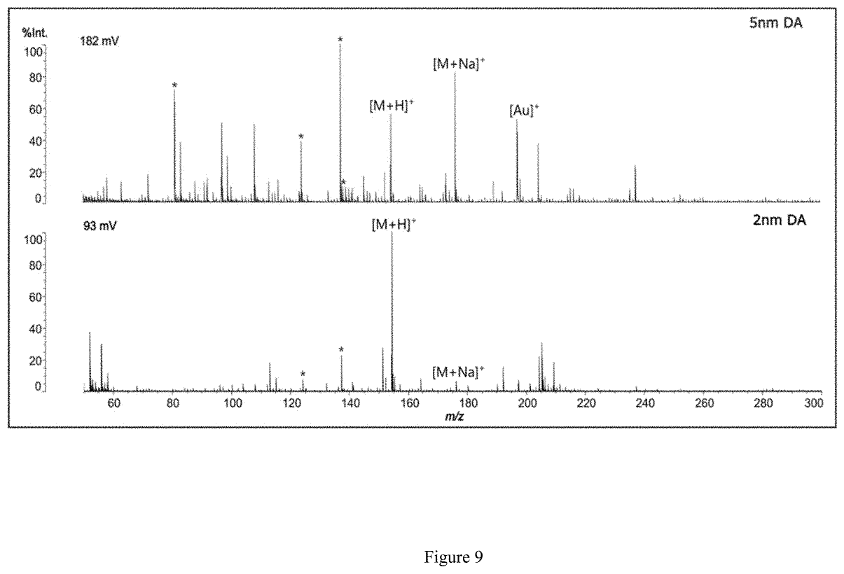

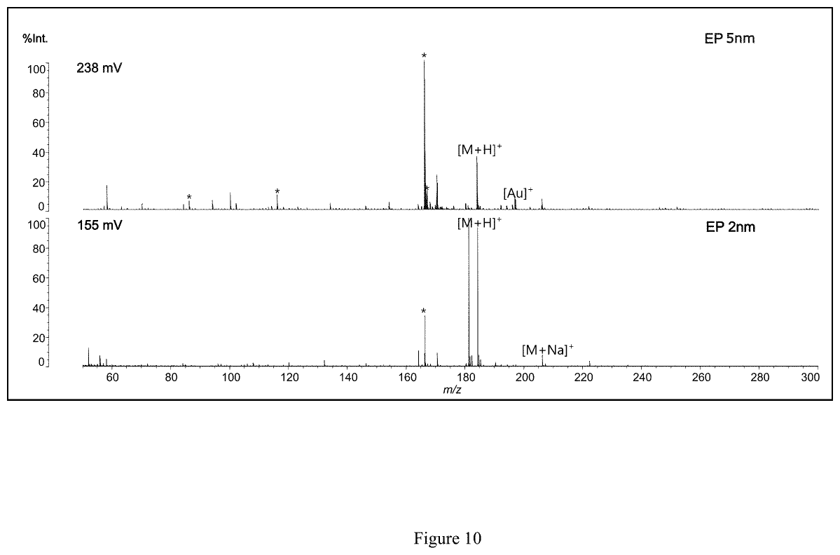

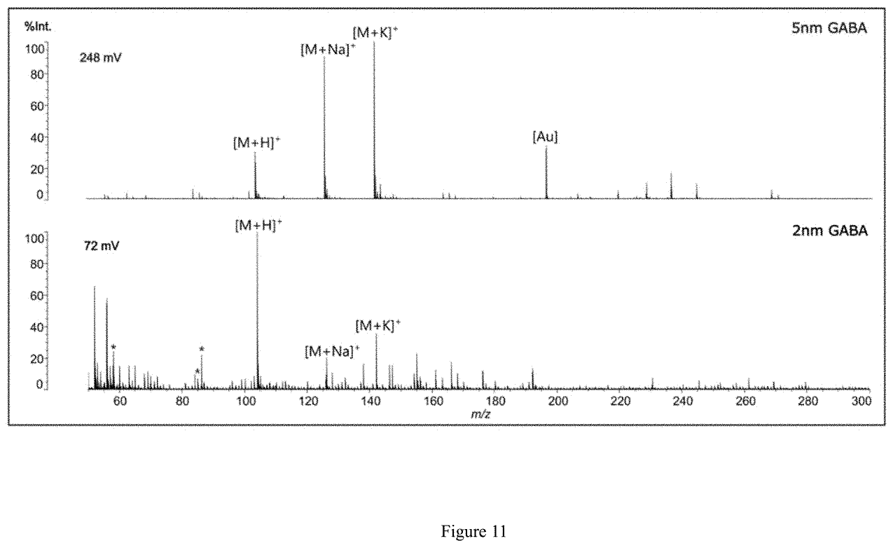

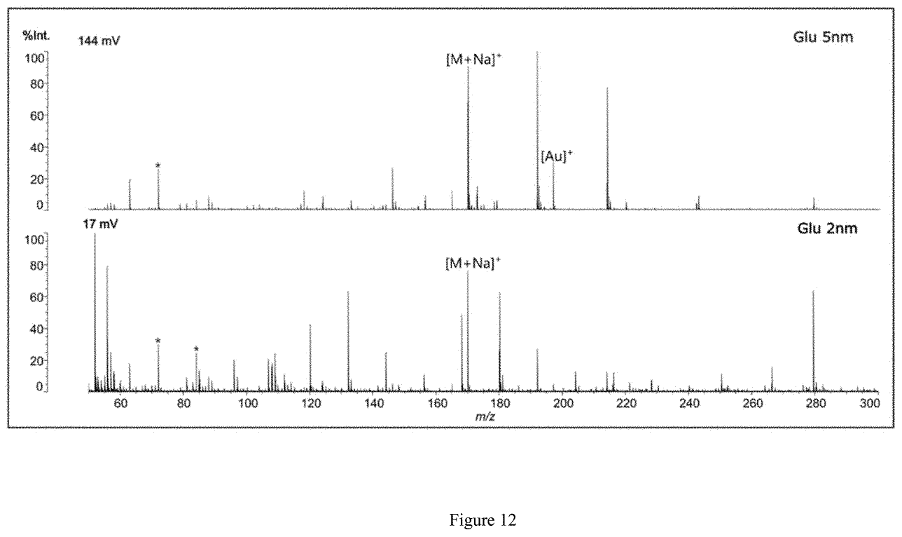

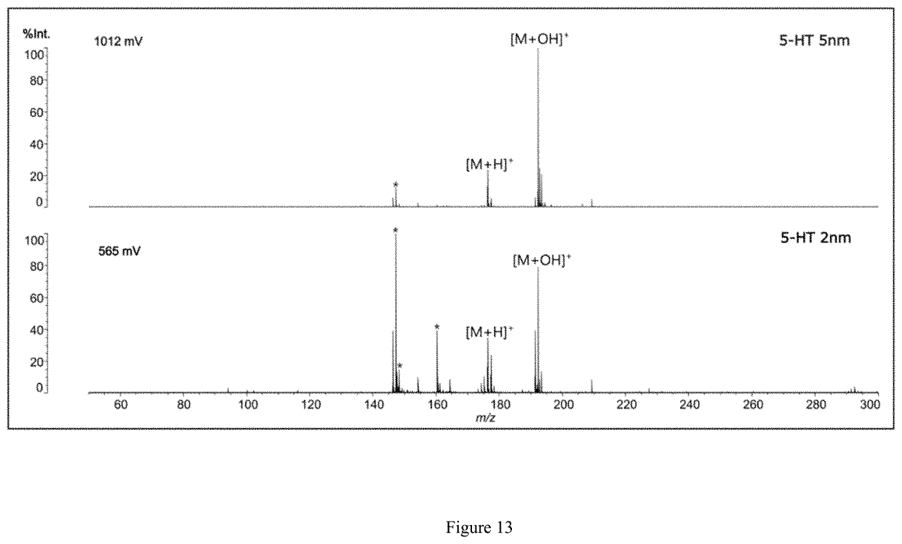

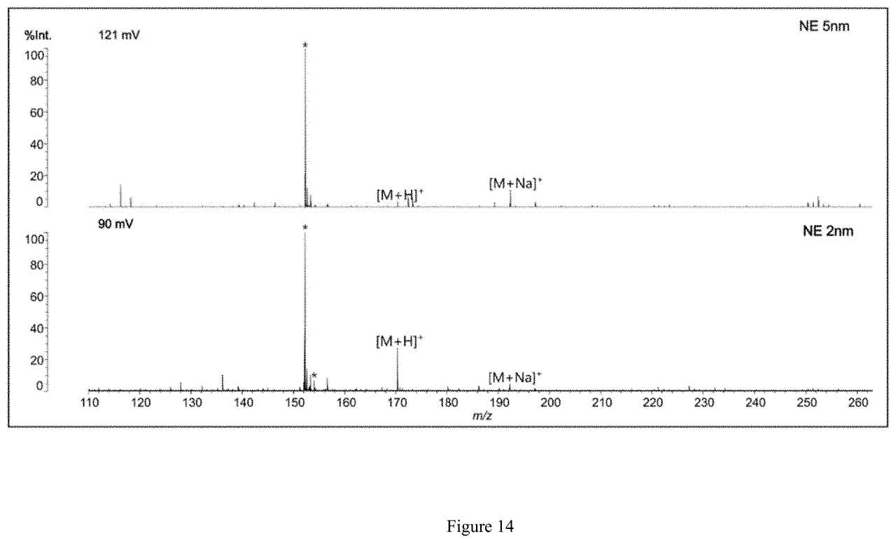

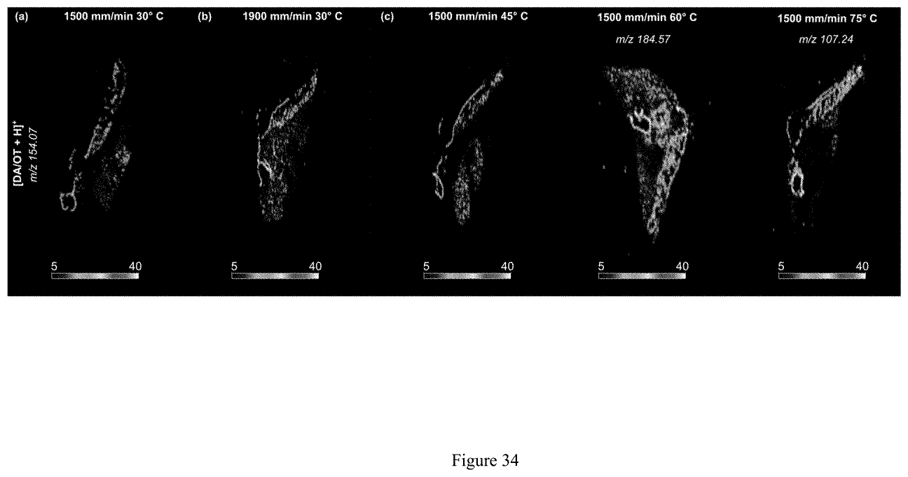

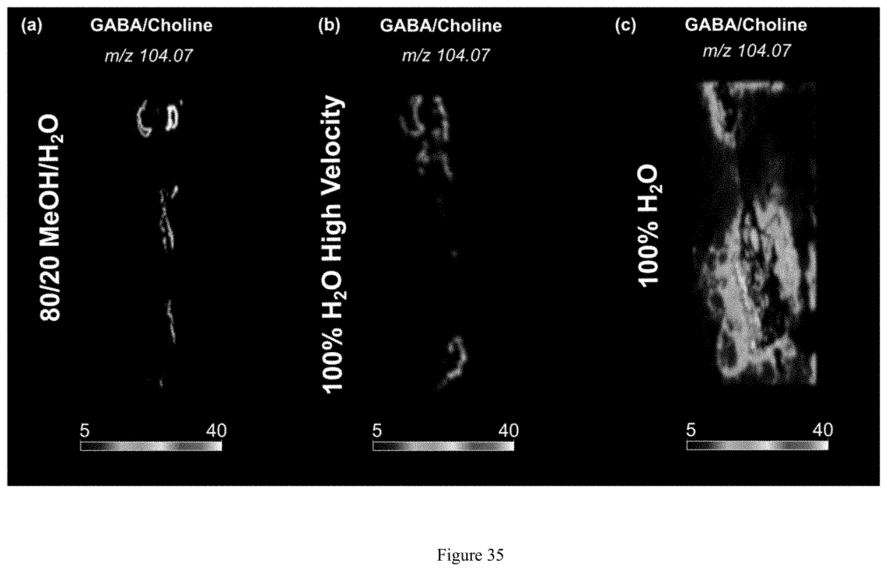

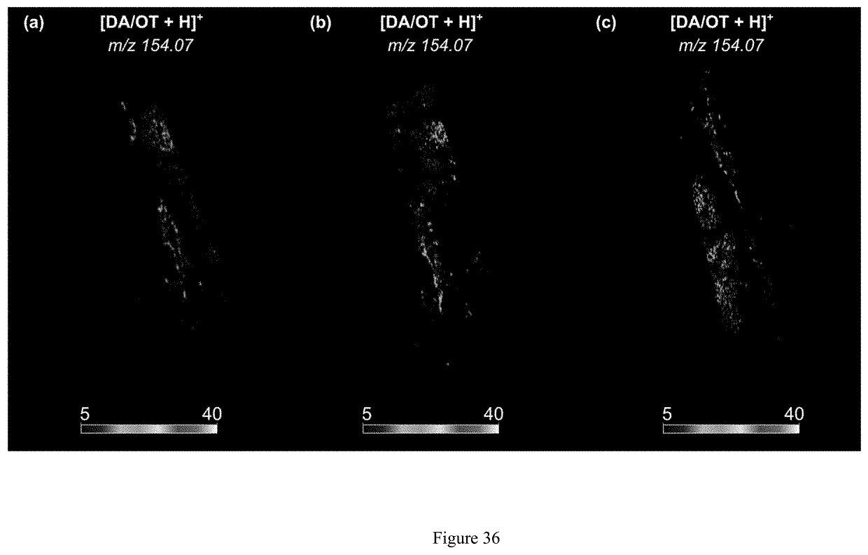

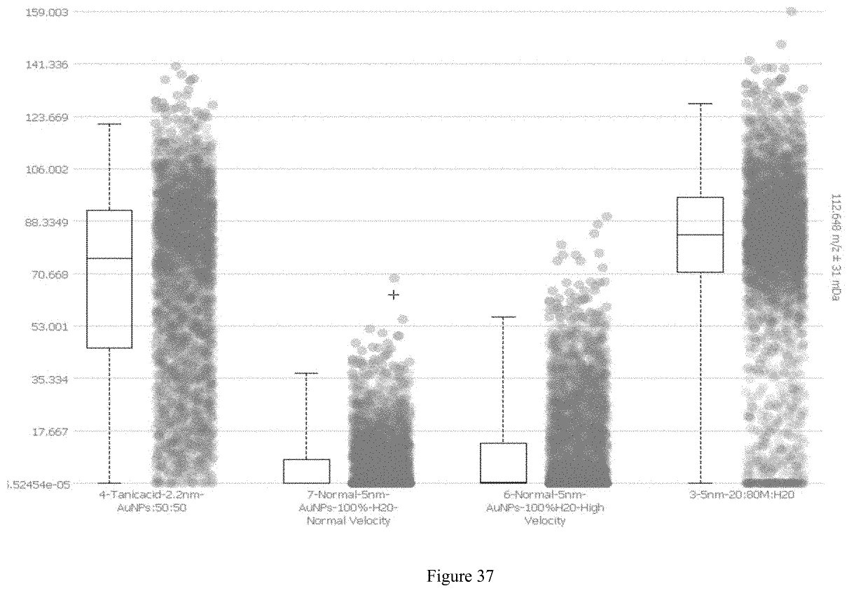

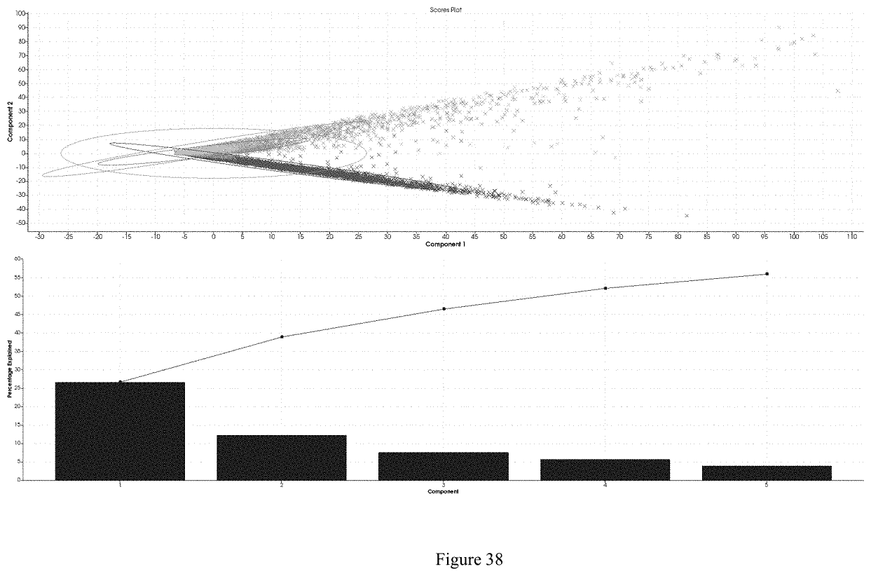

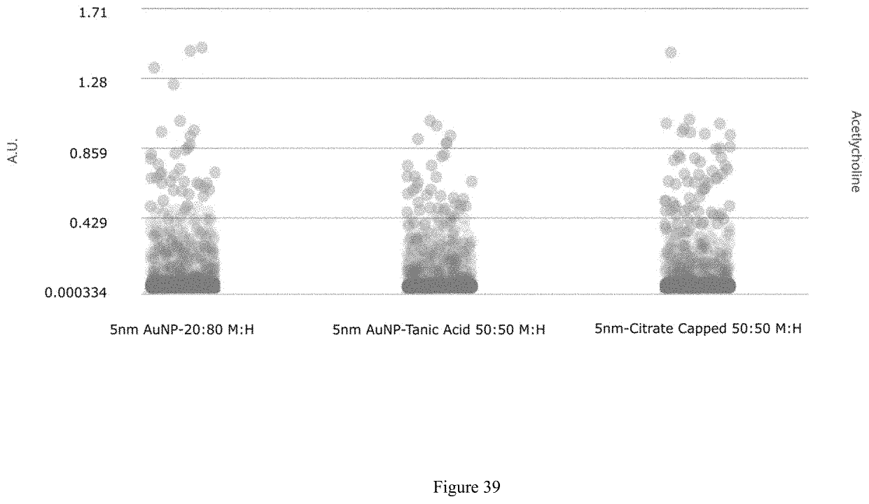

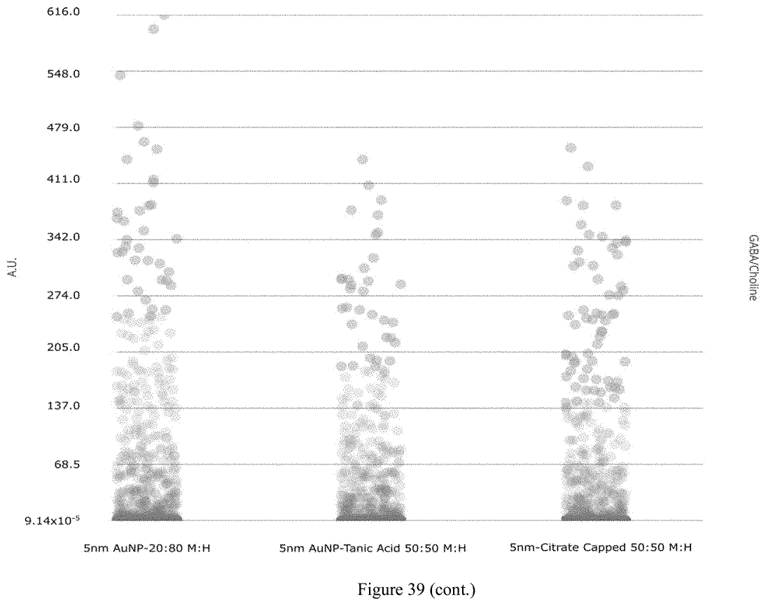

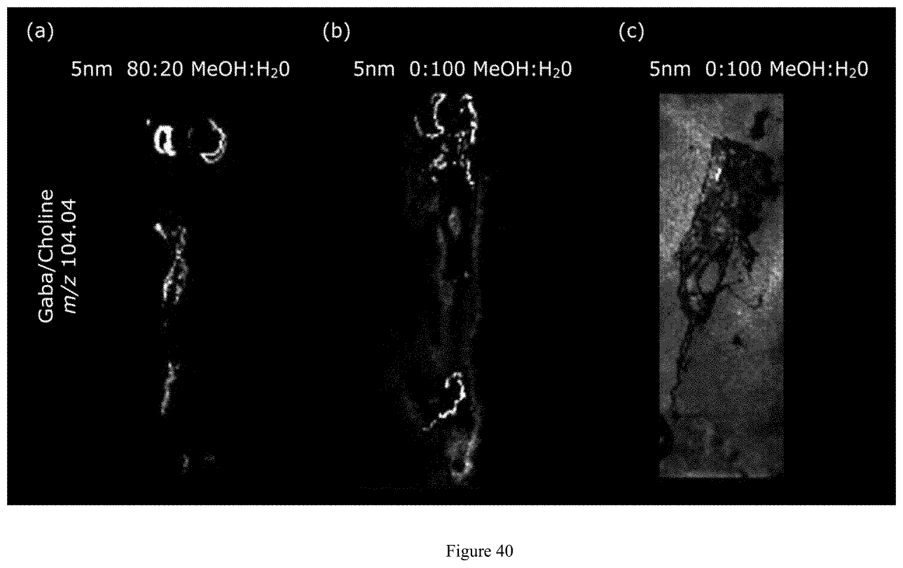

A series of experiments were conducted to determine if gold nanoparticles could facilitate imaging of biomolecules that were known to be difficult to image. Initially target plates were used to image a variety of different biomolecules and the results obtained are shown (FIGS. 8-14). Given these encouraging results using nanoparticles experiments were conducted using complex biological matrices such as serum or tissue homogenate (FIGS. 1 and 2). Following the success of these experiments a whole zebrafish was attached to a target plate and AuNPs dried on top (FIG. 34). These experiments led to the use of pneumatic spraying. In particular, the examples below illustrate experiments leading to the realization that pneumatic spraying of nanoparticles offered the ability to image a wide variety of neurotransmitters and metabolites in situ.

### Materials

The following were purchased from Millipore-Sigma (St. Louis, Mo.): α-cyano-4-hydroxycinnamic acid (CHCA), acetonitrile, acetylcholine chloride, ammonium bicarbonate, 2,5-dihydroxybenzoic acid (DHB), dopamine hydrochloride, epinephrine hydrochloride, gamma-aminobutyric acid, glutamic acid monosodium salt, norepinephrine bitartrate, octopamine hydrochloride, poly-D-lysine, serotonin hydrochloride, sinapinic acid (SA), tricaine methanesulfonate, and HPLC grade water and methanol. All reagents were ACS grade or higher, unless noted. Gold nanoparticles (AuNPs) with nominal size of 2 and 5 nm were purchased from Ted Pella, Inc. (Redding, Calif.). Sterile-filtered human serum was purchased from Sigma-Aldrich (St. Louis, Mo.). Young frozen rabbit brains stripped of meninges were purchased from Pel-Freeze (Rogers, Ak.). There were no additional safety considerations outside of normal chemical hygiene procedures.

### Crayfish Brain Preparation

Previously dissected and frozen crayfish brains (Procambarus clarkii) from Carolina Biological Supply (Burlington, N.C.) were thawed to room temperature and were mixed with 250 μL of pH 7.0 buffered 25 mM ammonium bicarbonate (ABC) and manually homogenized using a mortar and pestle. Next, another 250 μL ABC was added and the sample split and transferred to 2 test tubes which were spun at 8000 rpm for 8 min in a 3000 MWCO spin filter (EMD Millipore, Burlington, Mass.). The flow through was retained and spun again at 8000 rpm for 10 min and then 14000 rpm for 5 min. The remaining solution was distributed into 100 μL tubes and AuNPs added; the empirically determined the optimum AuNP-to-analyte ratio was for 5 μL of 2 nm AuNPs to be added to the 100 μL of sample.

### Zebrafish Husbandry

Adult zebrafish (Danio rerio) were purchased from Carolina Biological Supply (Burlington, N.C.), bred, and embryos collected. Within 4 hours-post fertilization embryos were transferred to Petri dishes containing embryo medium (E3) and kept at 28° C. E3 buffer was changed daily until 5 days-post fertilization when embryos were sacrificed using a 600 mg/L solution of tricaine methanesulfonate. All animal handling procedures were approved by IACUC #9-19 at the University of Scranton.

### Neuroblastoma Preparation

Neuroblastoma cells (SK-N-SH, HTB-11) were purchased from ATCC (Manassas, Va.) and kept frozen until use. Cells were cultured in Dulbelco's Modified Eagle Medium in a glass petri dish and across cleaned indium tin oxide (ITO) slides (Delta Technologies, Loveland, Colo.) coated with poly-D-lysine. Neuroblastoma cells were placed in a desiccator for 5 minutes before spraying with AuNPs (spraying details below).

### Sample Preparation

Aqueous NT solutions were prepared at 1 mg/100 μL. Using 2 nm and 5 nm AuNPs individually, samples had a final ratio of 1 AuNP:105 analyte molecules; samples were plated using the dried droplet method. Traditional matrices of CHCA, DHB, and SA were mixed with analyte molecules at a ratio of 10⁵ matrix:1 analyte and spotted on target plates using the dried droplet method. Human serum samples were prepared at a concentration of 1 mg/10 μL and an appropriate ratio of AuNPs was placed into solution. NT spiked organic matrix samples were prepared by taking the same starting serum sample, adding 5 μL of previously prepared NT solution (including matrix) and plating using the dried droplet method.

### LDI and MALDI MS Analysis

All target-plate based experiments were performed on a Kratos Axima MALDI-TOF MS (Shimadzu Scientific Instruments, Columbia, Md.). Conditions were optimized in positive ion reflectron mode, using pulsed extraction with a N₂ laser at 337 nm. Similar instrument conditions were used for traditional matrix and AuNP experiments. In general, an increase in laser power is needed for the AuNP samples compared to traditional matrices, with little difference in power for 2 nm and 5 nm AuNPs. Target-plate based experiments were repeated for efficacy on a Bruker Rapiflex MALDI TOF/TOF mass spectrometer (Bruker Daltonics Instruments, Billerica, Mass.) in reflectron positive ion mode, with a Nd:YAG laser at 355 nm. Since the data obtained were similar to previous target-plate experiments on the Shimadzu Axima, no target-plate data from this instrument are shown here. Note this is the first demonstration across instruments (i.e., lasers) for this method. MS/MS experiments were done on a Bruker Rapiflex MALDI TOF/TOF instrument in profiling mode using an M5 flat laser with 114 μm resultant field with 4000 laser shots and argon gas collision induced dissociation (CID). All measurements were completed with a 1 Da isolation window. Measurements were per-formed on tissue from the head region of the zebrafish embryo. MS/MS spectra of pure NTs were measured on a stainless steel target plate using the same method.

### Mass Spectrometry Imaging

Zebrafish embryos were placed in a 10 mm×10 mm×5 mm biopsy cryomold (Ted Pella) and embedded in Thermo Scientific Shandon™ M1 embedding media (Thermo Fisher Scientific, Waltham, Mass.). After freezing, the block was sectioned at 10 μm thickness at −16° C. and thaw-mounted onto cleaned ITO slides. Fresh frozen rabbit brain was sectioned at −20° C. without embedding in media at 10 μm thickness and thaw-mounted onto cleaned ITO slides. All cryo-sectioning was done on a Leica CM1860 cryostat (Buffalo Grove, Ill.). A traditional organic matrix preparation was performed using 10 mg/mL DHB in 50% MeOH/50% water and sprayed using an HTX M5 sprayer (HTX Technologies LLC, Chapel Hill, N.C.) with a nozzle temperature of 85° C. Eight passes were sprayed at a flow rate of 0.075 m/min with no drying time. Using the HTX M5 sprayer, 2 nm AuNPs were sprayed at either 30° C. or 45° C. One pass was sprayed at a flow rate of 0.010 m/min with 2 seconds drying time. All imaging experiments were performed on Bruker Rapiflex MALDI-TOF/TOF mass spectrometer. Spectra were obtained in positive-ion mode with 200 laser shots per pixel.

### Data Processing

All data was converted to imzML using flexImaging version 5.0 (Bruker Daltonics). The imzML files were then converted in msiQuant (Kallback et al., Anal Chem, 2016, 88, pp 4346-4353) for analysis and processing. RStudio was also used for data analysis. The package Cardinal (Bemis et al., Bioinformatics, 2015, 31, pp 2418-2420) under the Bioconductor Normalization was performed using total-ion-count (TIC) method. Regions of interest were selected by hand.

### Results

NTs analyzed include acetylcholine (ACh), dopamine (DA), epinephrine (EP), 4-amino butyric acid (GABA), glutamine (GLU), norepinephrine (NE), octopamine (OT), and serotonin (5-HT). Table 1 details the species observed for individual NTs using 2 nm and 5 nm AuNPs, along with FIGS. 8-14 showing mass spectra for all NTs listed. ACh on the target plate did not require AuNPs to ionize (attributed to the quaternary amine with permanent charge, see FIG. 8), but did on tissue. While these control experiments were done to determine if any NTs desorb as preformed ions, differences between target plate and on tissue were expected. It is inferred the differences in analyte concentration and the overall complexity of the background matrix affected ionization efficiency. Additionally, salt adducts were present, with fewer being observed for 5 nm as compared to 2 nm AuNPs; salt adducts are expected due to the high concentration of Na⁺ and K⁺ from the AuNP solution.

TABLE 1
Individual NTs observed species and mass accuracy.

| | | | Mass | | Mass |
|---|---|---|---|---|---|
| | Species and | 2 nm observed | accuracy | 5 nm observed | accuracy |
| Analyte | monoisotopic mass | species and mass | (ppm) | species and mass | (ppm) |
| ACh | M⁺⁻ 146.12, | M⁺⁻ 146.0, | 821, | M⁺⁻ 146.1, | 137, |
| | [M + H]⁺ 147.13 | [M + H]⁺ 147.0 | 883 | [M + H]⁺ 147.1 | 204 |
| DA | [M + H]⁺ 154.08, | [M + H]⁺ 154.0, | 519, | [M + H]⁺ 154.1, | 130, |
| | [M + Na]⁺ 176.07, | [M + Na]⁺ 176.0, | 398, | [M + K]⁺ 192.0 | 208 |
| | [M + K]⁺ 192.04 | [M + K]⁺ 192.0 | 208 | | |
| OT | [M + H]⁺ 154.08, | [M + H]⁺ 154.1, | 130, | [M + H]⁺ 154.1, | 130, |
| | [M + Na]⁺ 176.07, | [M + Na]⁺ 176.1 | 170, | [M + Na]⁺ 175.9, | 965, |
| | [M + K]⁺ 192.04 | | | [M + K]⁺ 191.9 | 729 |
| EP | [M + H]⁺ 184.10, | [M + H]⁺ 184.0, | 543, | [M + H]⁺ 184.0, | 543, |
| | [M + Na]⁺ 206.08, | [M + Na]⁺ 206.1 | 97 | [M + Na]⁺ 206.1 | 97 |
| GABA | [M + H]⁺ 104.07, | [M + H]⁺ 103.9 | 1633, | [M + H]⁺ 104.0, | 673, |
| | [M + Na]⁺ 126.05, | [M + Na]⁺ 126.0, | 397, | [M + Na]⁺ 125.9, | 1190, |
| | [M + K]⁺ 142.02 | [M + K]⁺ 142.0 | 141 | [M + K]⁺ 141.9 | 845 |
| GLU | M⁺⁻ 146.07, | M⁺⁻ 145.9, | 1163, | M⁺⁻ 145.9, | 1163, |
| | [M + H]⁺ 147.08 | [M + H]⁺ 146.9 | 1224 | [M + H]⁺ 146.9 | 1224 |
| NE | M⁺⁻ 169.07, | M⁺⁻ 168.9 | 1005, | M⁺⁻ 169.0, | 414, |
| | [M + H]⁺ 170.08, | [M + H]⁺ 169.9, | 1058, | [M + H]⁺ 169.9 | 1058, |
| | [M + Na]⁺ 192.06 | [M + Na]⁺ 191.9 | 833 | [M + Na]⁺ 191.9 | 833 |
| 5-HT | M⁺⁻ 176.09, | M⁺⁻ 175.9, | 1079, | M⁺⁻ 176.0, | 511, |
| | [M + H]⁺ 177.10, | [M + H]⁺ 177.0 | 565 | [M + H]⁺ 177.0, | 565, |
| | [M + Na]⁺ 199.08 | | | [M + Na]⁺ 199.0 | 402 |

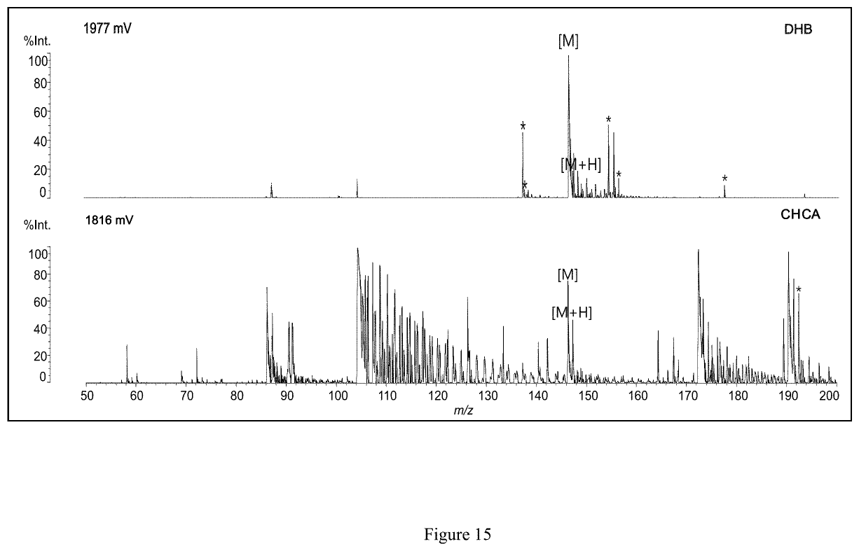

All pure compounds tested using AuNPs resulted in a reduction in background chemical noise as compared to organic matrices, which aided detection of the NTs. Detection of NTs with AuNPs was compared to that with all-purpose organic matrices (i.e., DHB or CHCA, see FIG. 15), which were unable to provide conclusive results for many of the NTs. Specifically, the use of DHB resulted in overlapping analyte peaks and matrix background chemical noise (e.g., 5-HT, DA, EP, GLU, and OT); i.e., analyte and matrix peaks could not be distinguished because of multiple isobaric species.

To show the efficacy of AuNPs to ionize NTs from a complex mixture, homogenized crayfish brains were analyzed. This is a particularly difficult sample to analyze using MS due to the small chemical footprint of the target molecules. AuNPs ionized many of the NTs typically found in a crayfish brain. Specifically, 2 nm AuNPs ionized DA/OT, EP, NE, 5-HT, ACh, GLU, and GABA/choline (see FIG. 1, Panel A) and 5 nm AuNPs ionized ACh, DA/OT, EP, GABA/choline, GLU, 5-HT. Compared to analysis with DHB (FIG. 1, Panel B), more NTs were observed with AuNPs and there was no overlap of matrix with potential NT signals (labeled as *); additionally, the optimized instrument conditions yielded a higher ion detection of 680 mV for AuNP compared to 144 mV with DHB. Analysis with SA (FIG. 1, Panel C) resulted in very high baseline noise which arises from needing a higher laser power to observe any signal at all and therefore overall ion detection was 1382 mV, but with only two NTs observed. Chemical background noise is still present with AuNPs, but is improved from organic acid matrices. Traditional methods of analysis for NTs in crayfish (and other vertebrate systems) typically include high performance liquid chromatography electrochemical detection (HPLC-ECD) which can be time consuming and require significant method development (Yang et al., Mol Neurodegener, 2011, 6, pp 6). This novel application of AuNPs could potentially transform the ability to analyze NTs in tissue homogenates of a variety of organisms commonly used in neuroscience research.

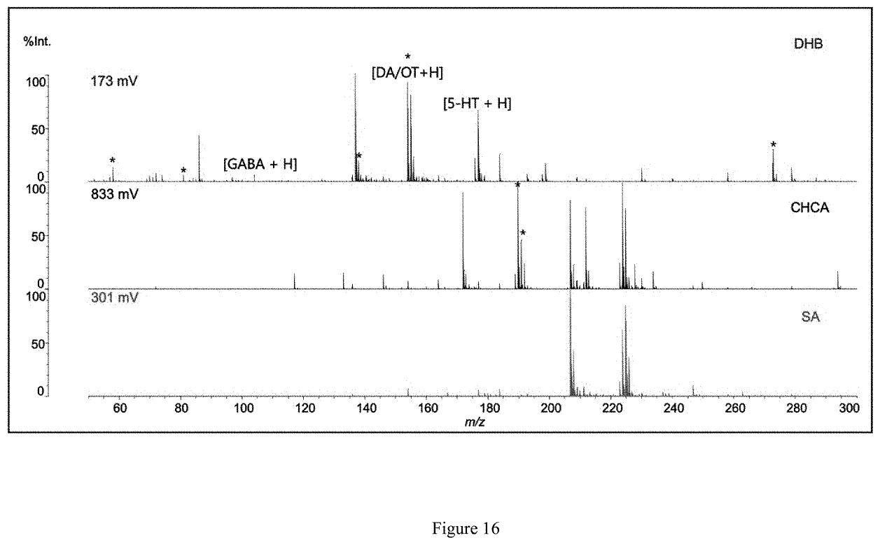

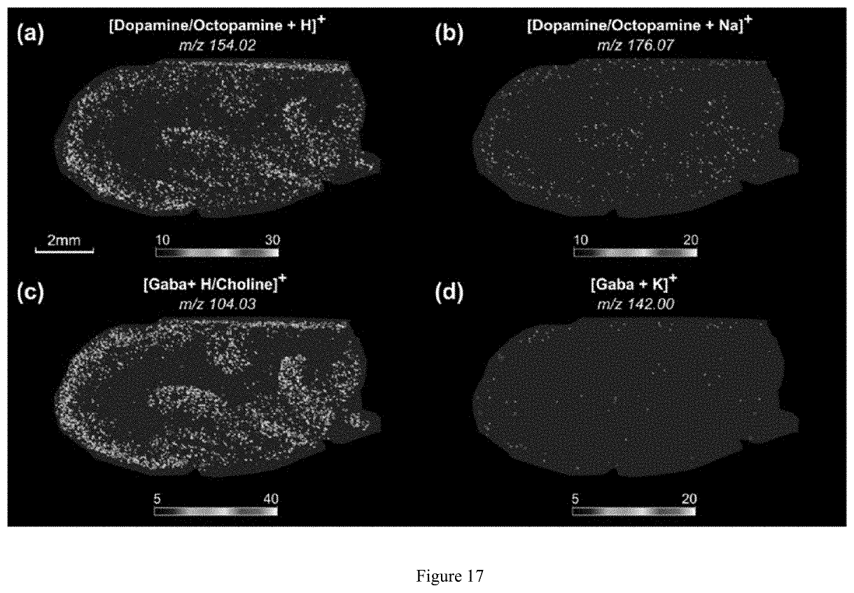

To further assess the utility of AuNPs for NT detection in biological samples, human serum was analyzed. Both 2 nm and 5 nm AuNPs ionized NTs at circulating physiological concentrations, for which many of the NT concentrations are in the 100s of pg/mg (Golabi et al., Medicine, 2016, 95, e5006 and Janik et al., Neurol Neurochir Pol, 2010, 44, pp 251-259). FIG. 2 shows 2 nm AuNPs ionizing GABA/choline, GLU, DA/OT, 5-HT, NE, and EP. This is the first known example of AuNPs ionizing NTs at a physiologically relevant concentration (e.g., the typical circulating concentration of DA in serum of 200 pg/mL (Golabi et al., Medicine, 2016, 95, e5006 and Janik et al., Neurol Neurochir Pol, 2010, 44, pp 251-259)). As an additional comparison, SA, CHCA, and DHB were run with spiked NTs in order to assess ionization suppression. All organic matrix spectra had significant chemical noise in the low mass region (see FIG. 16).

Although DHB may have produced signals for DA/OT and 5-HT it was not possible to distinguish these from matrix peaks. Also, to generate significant signal intensities for CHCA, high laser power was required, resulting in more spectral noise and poor spectral resolution. DHB also failed to ionize EP and GLU. CHCA did not ionize DA, EP, OT, and 5-HT. In contrast to organic matrices, both 2 nm and 5 nm AuNPs were able to ionize all NTs, highlighting the importance of matrix choice in LDI-MS when working in the mass range below m/z 300. Limits of detection (LOD) were determined on the target plate and concentrations as low as fmol/μL per spot were ionized using 2 nm and 5 nm AuNPs for all analytes. All resulting spectra were comparable within 2 orders of magnitude of analyte concentration and within 2 orders of magnitude of AuNP:analyte, as previously discussed (Sacks et al., J Mass Spectrom, 2018, 53, pp 1070-1077 and McLean et al., J Am Chem Soc, 2005, 127, pp 5304-5305) (see Methods section for details). Few previous LOD determinations of NTs have been done using LDI-MS. The closest comparisons for LDI-MS were an analysis of DA in the ng/mL range (Zheng et al., Anal Lett, 2016, 49, pp 1847-1861) and another study detecting NE and EP in the nmol/g range from tissue (Bucknall et al., J Am Soc Mass Spectrom, 2002, 13, pp 1015-1027). Additionally, electrospray ionization of select NTs has been reported in the nM range (of 5-HT, DA, and their metabolites (Suominen et al., PLoS One, 2013, 8, e68007)) and in the ng range (of 5-HT, DA, and their metabolites (Najmanova et al., Chromatographia, 2011, 73, pp 143-149)).

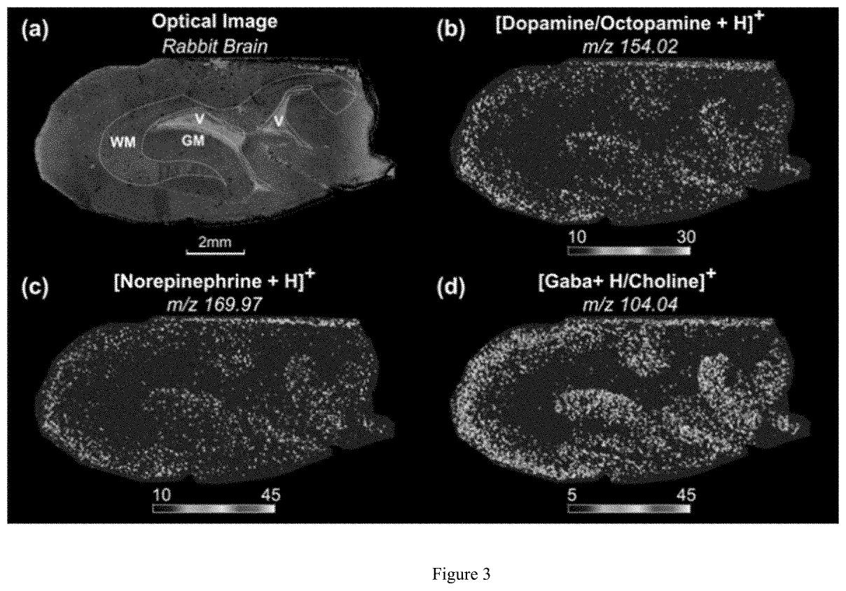

Next, the applicability of AuNPs for LDI-MSI was tested and coronal rabbit brain tissue sections (10 μm thickness) were examined as a proof-of-concept experiment. Given the similarity in performance of 5 nm and 2 nm AuNPs on the target plate, only 2 nm AuNPs were used from this point forward. The same NTs that were detected in target plate experiments were also observed in LDI-MSI; FIG. 3, Panel A shows the optical image of the rabbit brain section for reference, and FIG. 3, Panels B-D show the distribution of DA/OT, NE, and GABA/choline at 20 μm lateral spatial resolution. Importantly, the images show a difference between white and gray matter regions of the brain, with the visualization of the folds of the gray matter and the interior cavity of white matter that typically lacks NT signal. White matter contains axons, which are typically surrounded by the myelin sheath, gray matter contains most of the neuronal cell bodies, leading to an expected difference in NT abundance. GABA (Jensen et al., Biomed, 2005, 18, pp 570-576 and Choi et al., Neuroimage, 2006, 33, pp 85-93) DA, (Reader et al., Brain Res, 1979, 177, pp 499-513) and NE (Reader et al., Brain Res, 1979, 177, pp 499-513) were previously been shown to have differences in concentration in white vs. gray matter using magnetic resonance imaging techniques, though this has not previously been visualized using MSI. The ability to map NT location is useful for neurological research; for example, DA detection in white matter has been used for tracking the progression of disease in Huntington's (Ciarmiello et al., J Nucl Med, 2006, 47, pp 215-222) and Parkinson's disease (Haghshomar et al., Neuroscience, 2019, 403, pp 70-78 and Howe et al., Nature, 2013, 496, pp 498-503), and gray matter density informs on fibromyalgia (Wood et al., J Pain, 2009, 10, pp 47-52). Finally, tissue sections which contain fewer salt adducts than target plate experiments, resulted in images showing much lower intensity in Na⁺ and K⁺ adducts of NTs than those observed in target plate experiments (see FIG. 17). This could be attributed the to a change in how ionization occurs, with the soft desorption of protonated species being more favorable than that of the salt adduct; more experimentation to evaluate these differences is needed and ongoing.

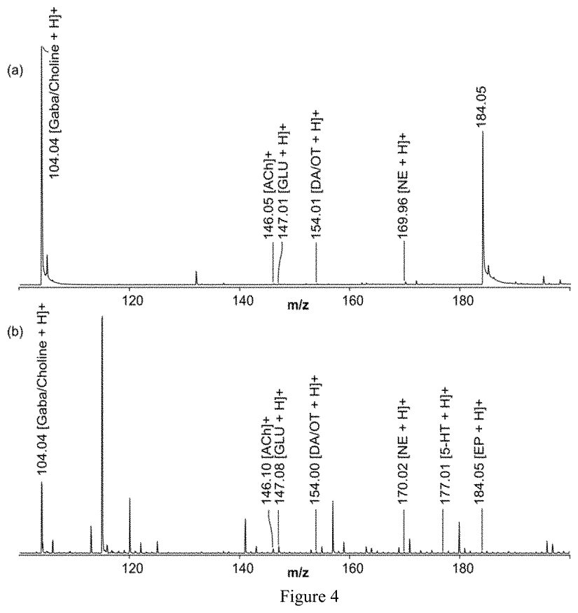

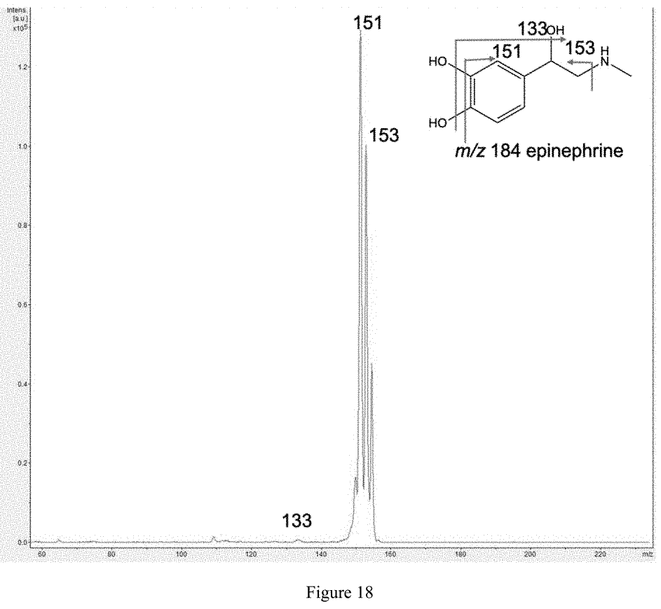

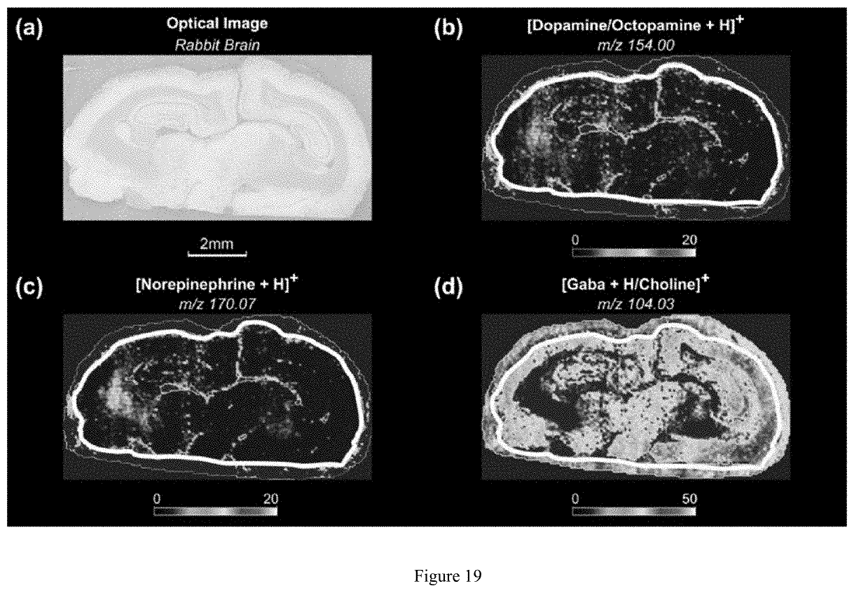

The mass spectrum for rabbit brain slices, which was normalized to total ion count (TIC), is shown in FIG. 4 with a comparison to tissue sprayed with CHCA. In FIG. 4 Panel A, near m/z 104 and 184 there is baseline distortion and there is an area where no additional peaks are observed, which could result from the high intensity of these two peaks. CHCA is known to extract lipids and m/z 184 is typically identified as the phosphatidylcholine headgroup or cytosolic phosphocholine, which also results in m/z 104 as choline as a decomposition product, or free choline in present in the cytosol (Murphy et al., Mass Spectrom Rev, 2011, 30, pp 579-599 and Van Hove et al., Cancer Res, 2010, 70, pp 9012-9021). For the AuNP samples, MS/MS of pure GABA and choline do not show any fragmentation differences and m/z 184 is confirmed as NE (see FIG. 18). Even with minimal spraying of organic matrix, the tissue is saturated with these two ions resulting in few identifiable peaks in the low mass range. Overall, AuNPs are advantageous for sample preparation and preservation of signal intensities. An additional improvement of using AuNPs for MSI is that there is minimal to no delocalization of analyte. FIG. 19 shows a typical CHCA spraying protocol on a rabbit brain tissue section and the resulting delocalization, where m/z signals extend beyond the tissue margins (bold white line).

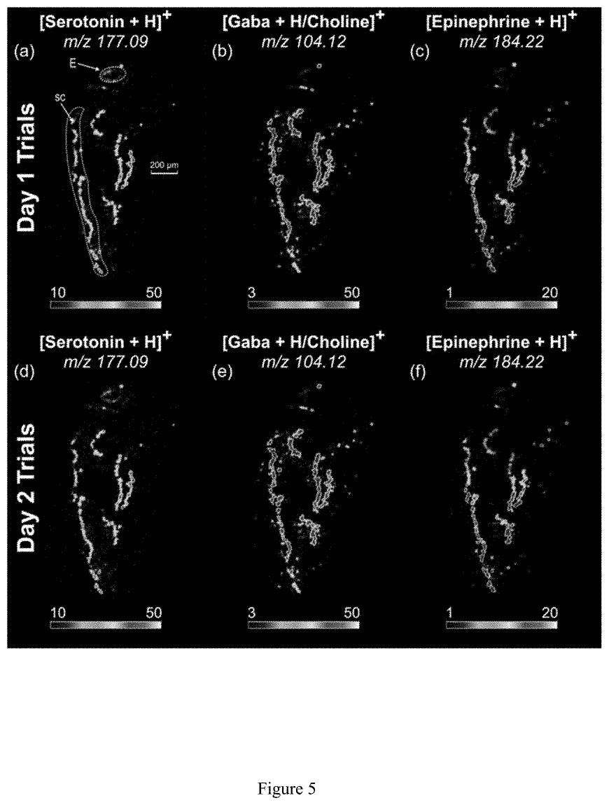

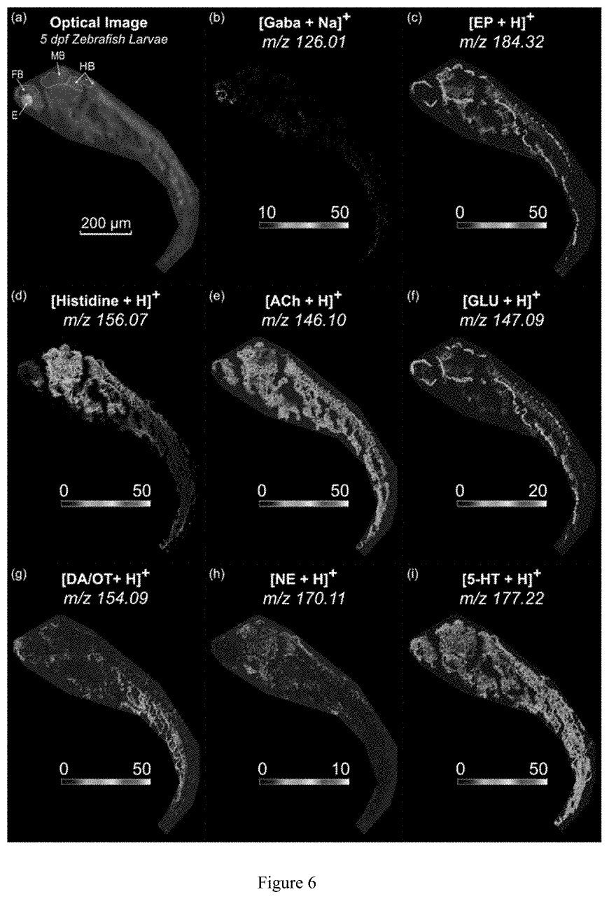

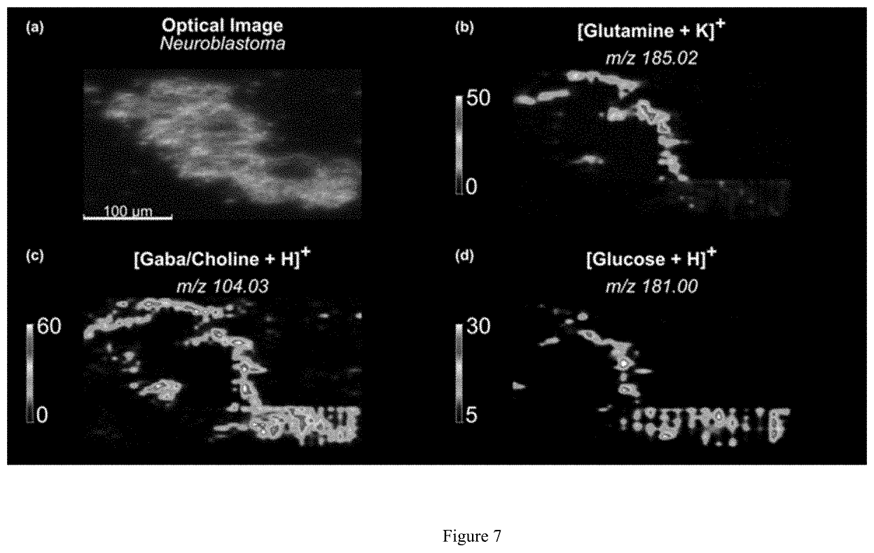

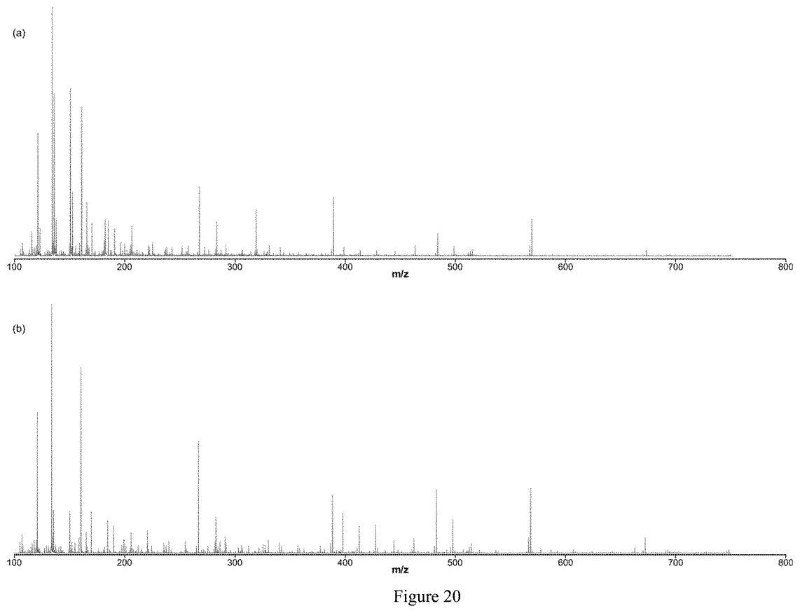

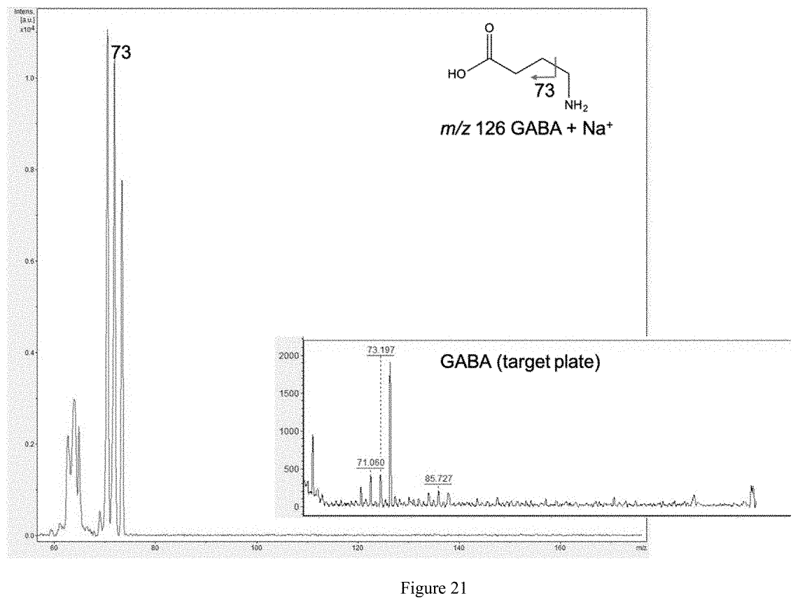

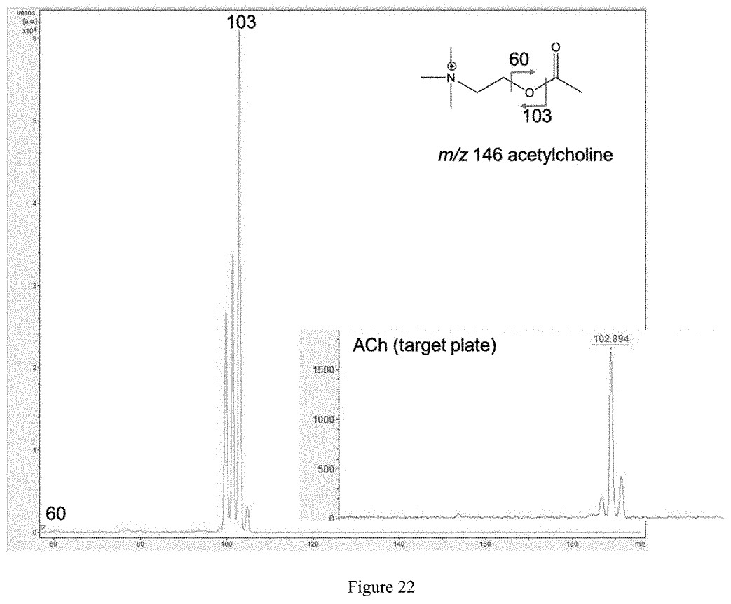

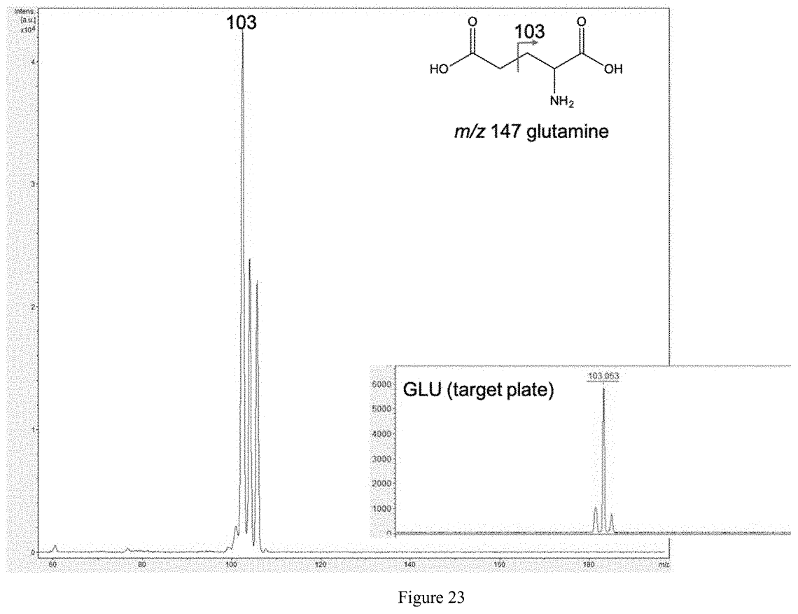

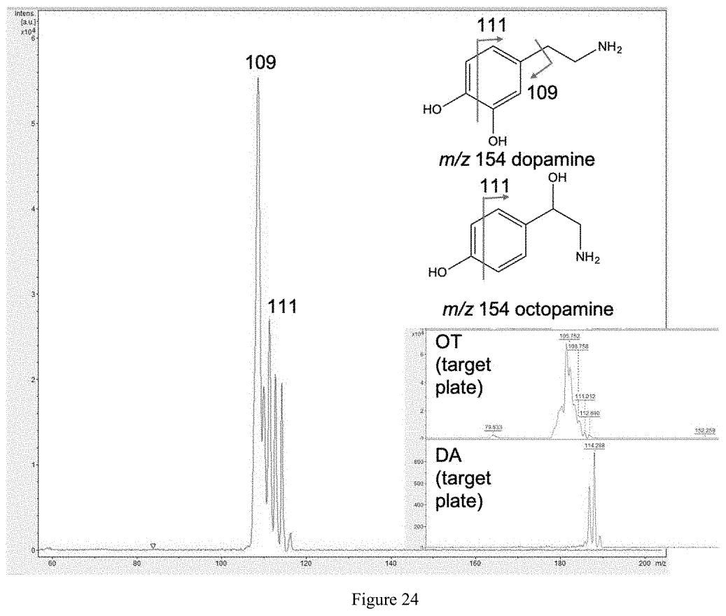

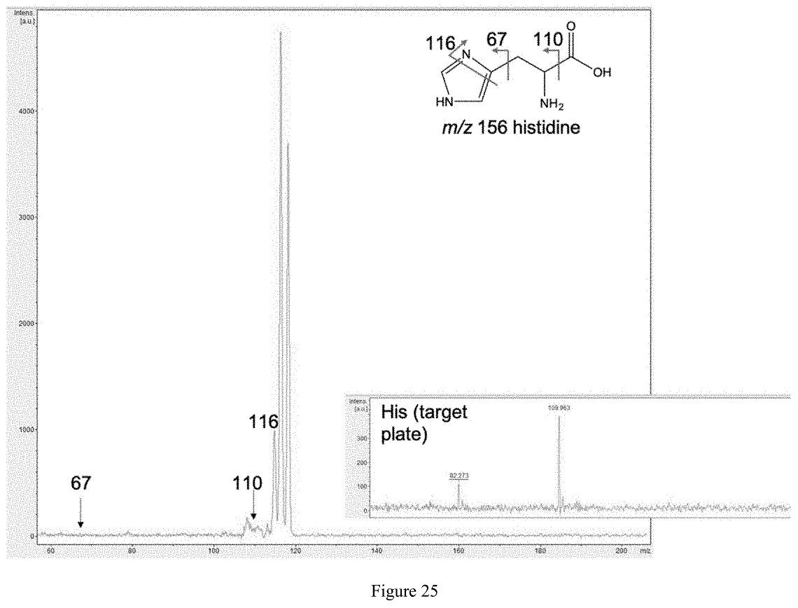

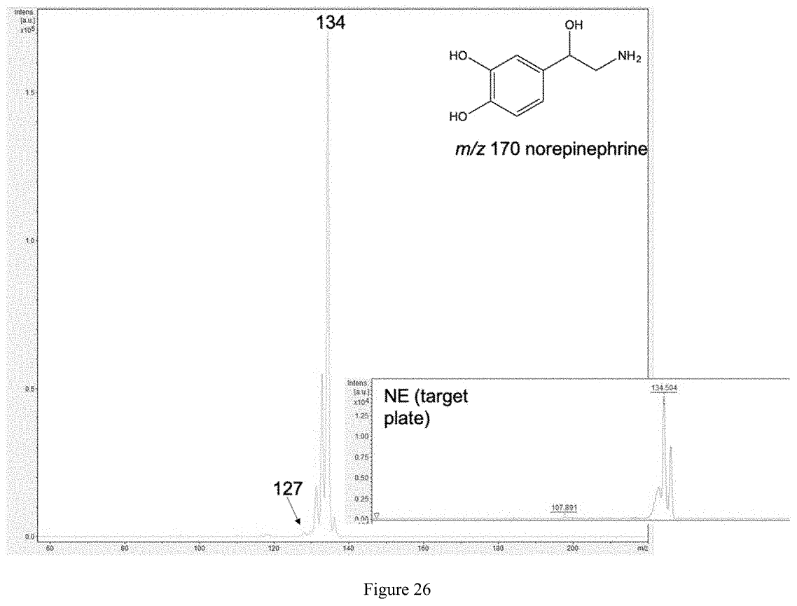

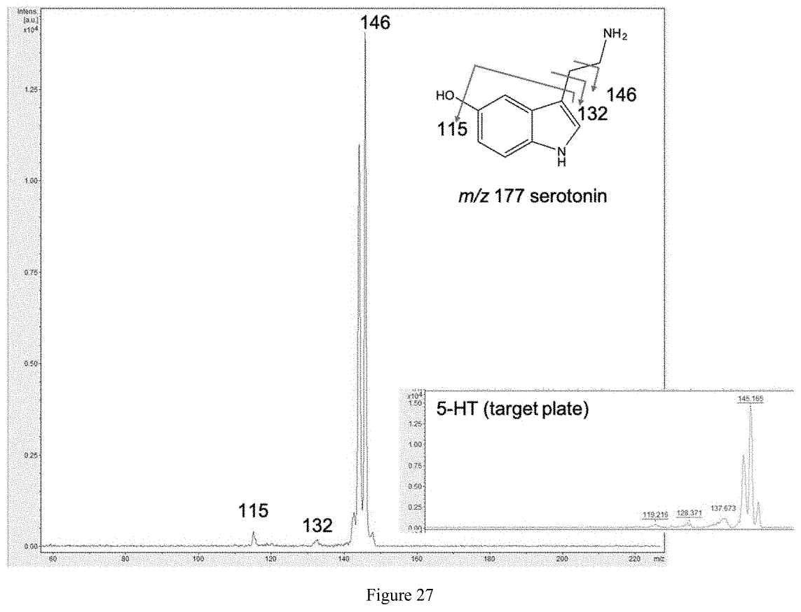

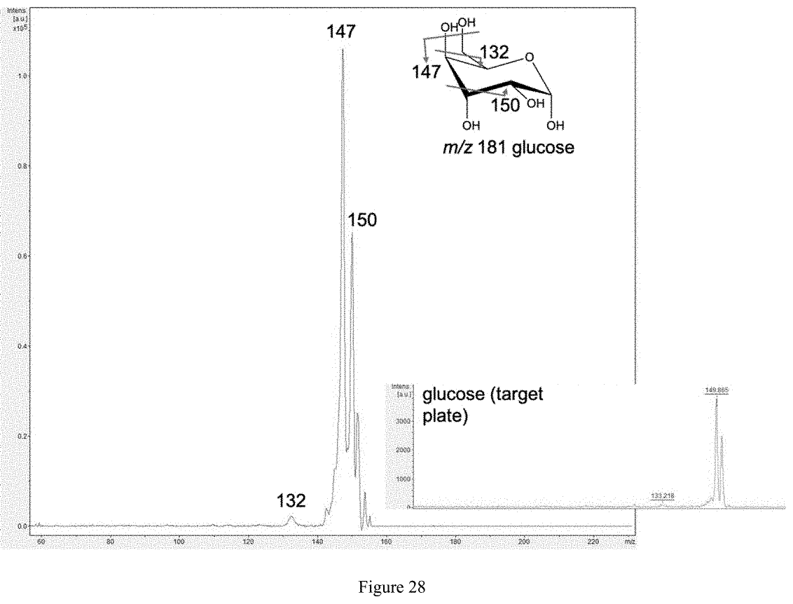

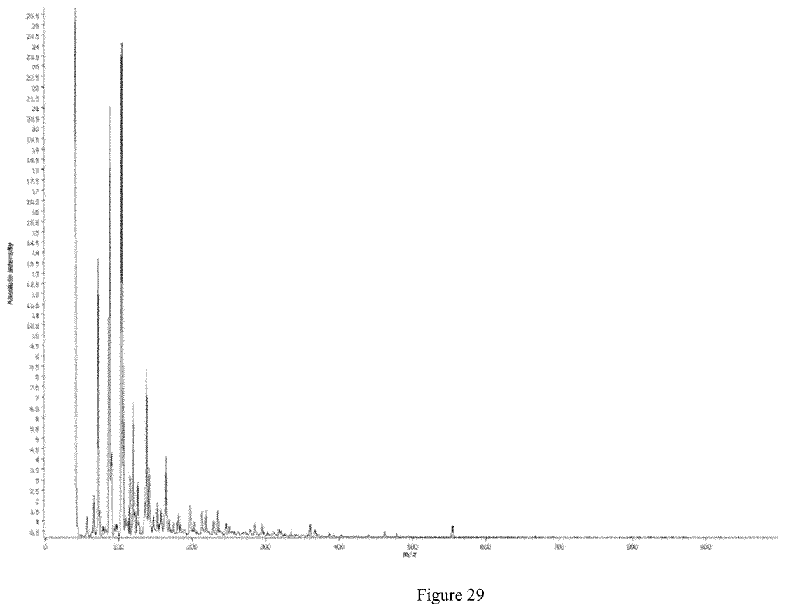

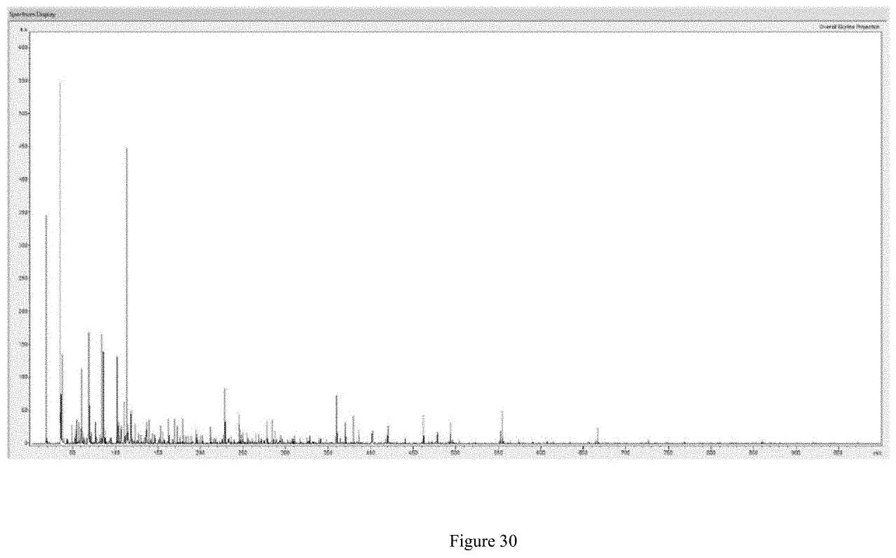

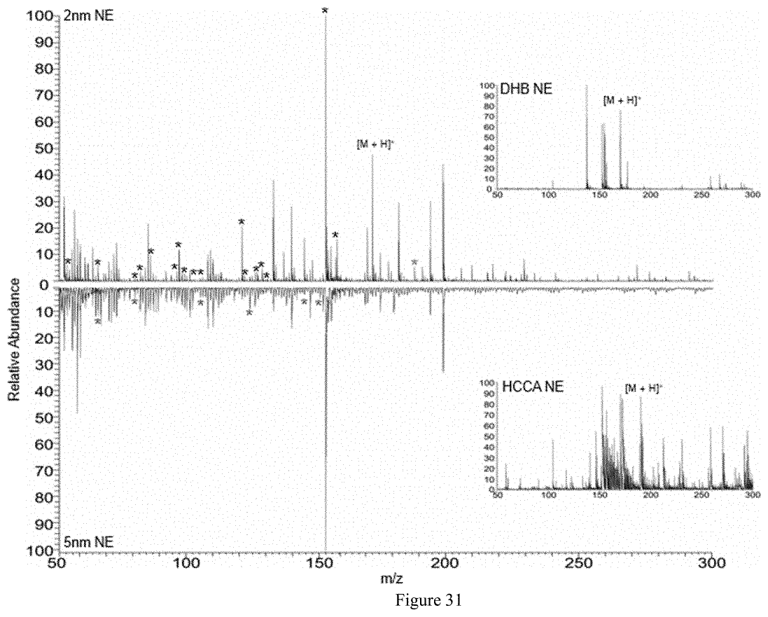

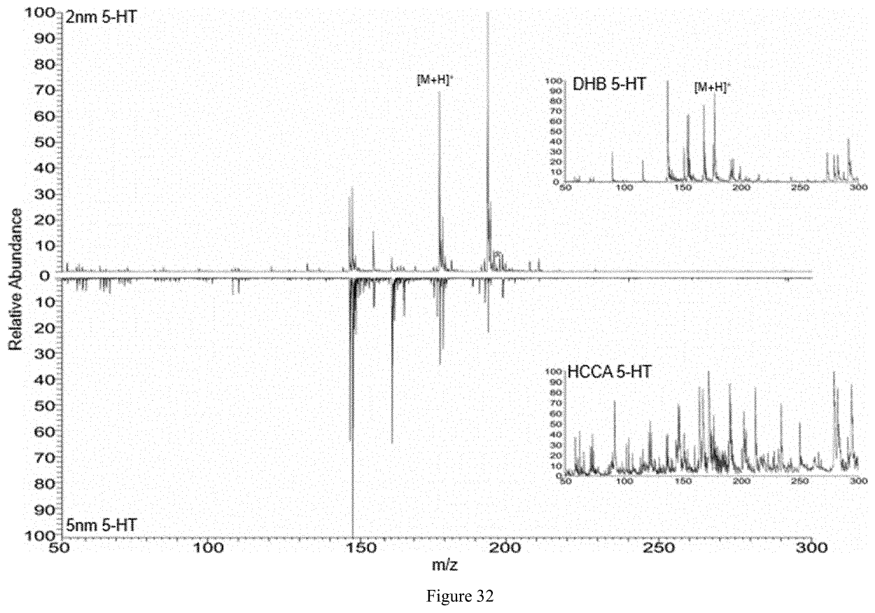

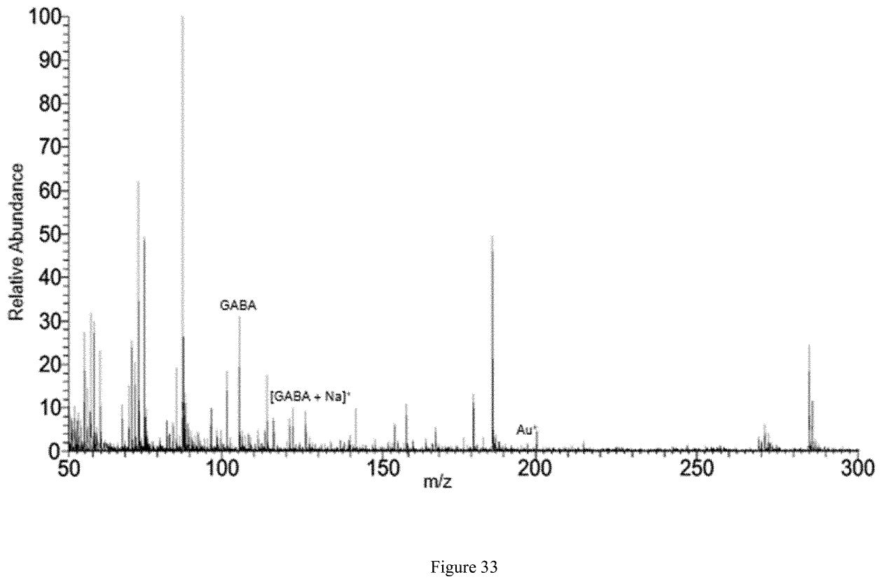

Expanding the utility of MSI to zebrafish embryos, which are 1-2 mm in size, presents several new challenges, including tissue preparation (e.g., mounting and cryo-sectioning) and if MSI can be achieved at high enough spatial resolution to adequately map the distribution of NTs. This organism is of interest because it is a widely accepted model for genetic and neuroscience studies owing to their similarities in neuroanatomy and development to higher level vertebrates, as well as conservation of metabolic pathways. The rapid breeding cycle, basic husbandry, and early morphology makes zebrafish an attractive model. FIG. 5 shows MSI data from 10 μm thick tissue sections of 5-day post-fertilization (dpf) zebrafish embryos imaged at 20 μm lateral resolution from an axial cryo-sectioning orientation, with spraying only one pass of AuNPs for sample preparation. All the previously characterized NTs were observed by MSI, with images of 5-HT, GABA/choline, and epinephrine shown in FIG. 5 (with embryo orientation of eye at the top and tail at the bottom). The neural tube (i.e., spinal cord) contains neural crest cells that migrate concomitantly with somites, followed by subsequent somite differentiation into the basal lamina (Raible et al., Dev Dyn, 1992, 195, pp 29-42); this allows for observation of the outline of the rapidly expanding somatic muscle that surrounds the spinal cord and notochord. FIG. 5 Panels D-F (bottom row) shows a subsequent imaging run performed on the tissue after storing the slide overnight at −20° C. There is no apparent difference in the NT spatial images after freezing the tissue and no additional application of AuNPs was required. The ability to acquire additional data on tissue allows for repeated runs and could significantly impact methods of data collection and the number of organisms required in research. Additional imaging runs (up to 8) were performed on multiple tissue areas and there was no discernable difference in the spatial distribution of NTs and intensity after repeated laser shots on the same area. FIG. 20 shows the average mass spectrum from the first and second imaging runs. Organic matrices typically require exacting conditions in order to effectively image small molecules and do not allow for repeat runs without washing and matrix re-application (Steven et al., Anal Bioanal Chem, 2013, 405, pp 4719-4728; Goodwin et al., J Proteomics, 2012, 75, pp 4893-4911; and Swales et al., Int J Mass Spectrom, 2019, 437, pp 99-112) yet it is demonstrated that there is extreme flexibility in storage of tissue when using AuNPs for MSI. For confirmation of the detected NT species in zebrafish, anatomical clues from a tissue atlas were used (Cheng et al., Penn State Bio-Atlas, http://zfatlas.psu.edu), computational data analysis methods (e.g., segmentation analysis) and MS/MS (see FIGS. 18 and 21-28). Thus far 10 precursor ions have been verified using MS/MS on one zebrafish tissue section with no loss of signal, suggesting more are possible. These are the one of the first MSI data of zebrafish embryos that simultaneously map multiple NTs, which presents exciting new opportunities for developmental biology research. The corresponding skyline mass spectrum from the MSI run is shown in FIG. 29. Note that the skyline spectrum is shown so that low intensity ions will have the same intensity as they appear at the individual pixel level even though they may only be present in a small fraction of the pixel spectra. The skyline spectrum was used so that a maximum number of signals could be interrogated to detect other small molecules of interest, possibly beyond neurotransmitters. A fully detailed examination of these data is ongoing. In addition to these proof-of-concept experiments on various biological samples and tissues, the limits of lateral spatial resolution on tissue using AuNPs for LDI MSI of zebrafish embryos are extended. FIG. 6 shows LDI imaging at 5 μm spatial resolution of sagittally cryo-sectioned 5 dpf zebrafish embryos. The scanned optical image in FIG. 6, Panel A provides anatomical references including the eye, forebrain, midbrain, hindbrain, and spinal cord. New molecules of interest are shown here including a taurine image at m/z 126.01 (FIG. 6, Panel B) and a histidine image at m/z 156.07 (FIG. 6, Panel D) as well as previously listed NTs including EP, ACh, GLU, DA/OT, NE, and 5-HT. With this higher spatial resolution exists the ability to discern anatomical features (e.g., brain, eye, neural tube) in much more detail than in FIG. 5. Specifically, the outline of the eye and the neural tube line are very apparent, as well as differentiation between forebrain, midbrain, and hindbrain. Blank spaces with no NTs detected likely correspond to the otic and pharyngeal cavities (Cheng et al., Penn State Bio-Atlas, http://zfatlas.psu.edu). Multiple NTs appear in the organ cavity, including the heart (Vargas et al., Zebrafish, 2017, 14, 106-117) and intestinal area (Njagi et al., Anal Chem, 2010, 82, pp 1822-1830), which have previously been established as sites of NT location in embryonic species. In addition to the previously discussed importance of NTs, histidine is a molecule of interest because it is a precursor to histamine which has important neuroprotective effects (Bae et al., Brain Res, 2013, 1527, pp 246-254) and it has specifically been shown to promote astrocyte migration after cerebral ischemia (Liao et al., Sci Rep, 2015, 5, pp 1-14). Lastly, GABA is an adduct of Na⁺ here at m/z 126, which distinguishes the protonated form from the overlapping signal of choline at m/z 104. Choline with a Na+ adduct would appear at m/z 63 (because it would be doubly charged), making this a clear way to distinguish the two NTs. The corresponding skyline mass spectrum from this MSI run is depicted in FIG. 30.

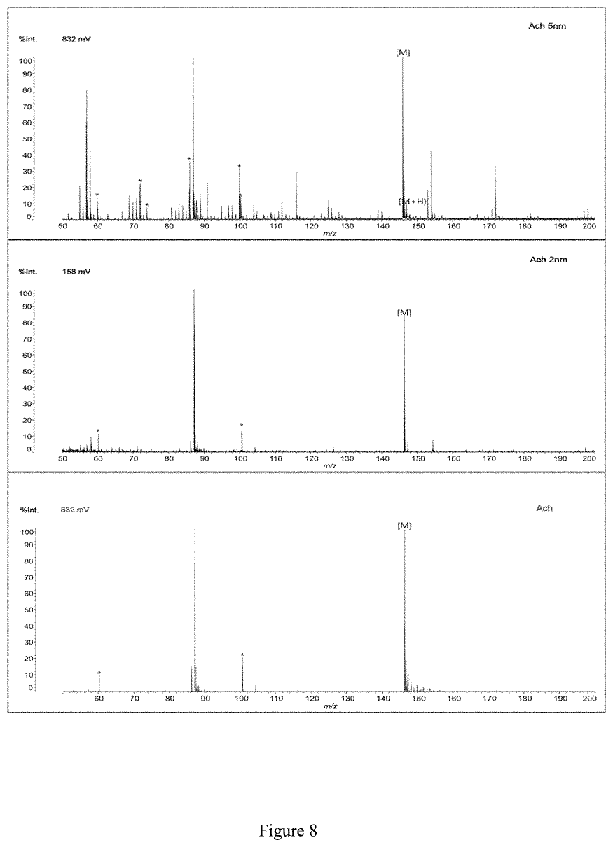

Continuing with the exploration of difficult-to-analyze samples, cells were imaged with the same approach. Previous examples of single-cell MSI have used only high-resolution instruments (Gilmore et al., Annu Rev Anal Chem, 2019, 12, pp 201-224) or transmission geometry based MALDI imaging (Niehaus et al., Nat Methods, 2019, 16, 925-931) in this demanding field of research. There are multiple challenges, including achieving a lateral spatial resolution that provides useful cellular information (<5 μm), achieving small enough matrix crystal sizes, and ionizing enough molecules for sufficient detection sensitivity. Previous studies have largely focused on lipids, whereas we have expanded single-cell MSI to small molecules. FIG. 7 shows neuroblastoma cells that were grown on ITO slides and then imaged at 5 μm. The optical image (FIG. 7, Panel A) shows the overall cell density within the imaged area and the molecules imaged are glutamine, GABA, and glucose. The entire rectangular panel was imaged in order to account for potential background signal from the growth media, but no significant signals were observed outside of areas containing a high-density of cells. The use of citrate-capped AuNPs that can be pneumatically sprayed onto tissues extends the analytical capabilities of LDI-MSI to compounds that have been difficult to ionize or must first be derivatized to be ionized and presents a highly time- and cost-effective preparation. While chemical derivatization strategies can target primary amines successfully, extensive synthesis and long preparation times are needed, and the cost can be prohibitive at a minimum of $7 per slide for the needed reagents. Even traditional organic matrices cost at least $1 per slide for reagents and take 2-3 times longer than an AuNP spraying protocol, which costs less than one thousandth of a cent per slide. The presented methods enable a broad range of new applications in neuroscience, pharmacology, drug discovery, and pathology.

### Example 2: Imaging of Neurotransmitters Using AuNPs with Laser Desorption Ionization Mass Spectrometry

### Methods

Neurotransmitter samples were prepared at a concentration of 1 mg/100 μL. Using 2 nm and 5 nm AuNPs individually, samples had a final ratio of 1 AuNP:10⁵ analyte molecules. A traditional dried droplet experiment was done for plating of samples. Limits of detection were determined for each analyte. This experiment was repeated using 2,5-dihydroxybenzoic acid (DHB) and a-cyano-4-hydroxycinnamic acid (HCCA). Zebrafish embryos were prepared by removal of the chorion and yolk sac, then mounted onto the MALDI plate. The embryos were mounted using an OCT embedding compound or by a thaw mounting process using liquid nitrogen. MS experiments were performed in reflectron-positive ion mode, on a Kratos Axima (Shimadzu, Columbia, Md.) which was equipped with a 337-nm nitrogen laser. Spectra were either generated using 256 laser pulses (shots) or 361 profiles with each profile containing 2 laser pulses. The lateral and vertical movement step sizes of the sample stage were set at 100 μm during the IMS experiments, thus generating a raster resolution of 100 μm.

### Results

5 nm AuNPs result in more fragment ions than 2 nm AuNPs. Also, fragmentation is more abundant with 5 nm AuNPs. AuNPs fragmentation is similar to Electron Impact mass spectra 2 nm AuNPs give unique [OH]. reactions not seen with DHB/HCCA and 5 nm AuNPs. It is theorized that the radicals arise from photochemical reactions from citrate.

### Example 3: Method Development for Using AuNPs in Mass Spectrometry Imaging

### Methods

MSI experiments were performed on zebrafish embryos that were embedded in M1 embedding media. Tissue was sectioned at 10 μm thickness and thaw mounted onto ITO slides at −16° C. AuNPs (Ted Pella; Redding, Calif.) or organic matrix (e.g., DHB) were sprayed onto tissue sections using an HTX Imaging Sprayer. All imaging experiments were performed on Bruker Rapiflex MALDI TOF/TOF in reflectron positive mode at various pixel sizes ranging between 5-200 microns. Sample preparation variables range from: (i) traditional matrix vs. AuNPs, (ii) AuNP size, (iii) spray parameters, and (iv) number of spray passes. Method development investigates the effects of the following: (i) NP concentration, (ii) number of spray passes, (iii) organic:aqueous solvent mixes, (iv) spray temperatures, (v) spray velocity, (vi) NP size and capping agent, and (vii) tissue handling and preparation.

### Results

Both 2 and 5 nm AuNPs increase ionization of desired analytes and decrease background noise for zebrafish embryos and rabbit brains. NPs result in greatly reduced signal delocalization and increased lateral spatial resolution capabilities, most likely owing to the lack of need for matrix crystallization. Spraying parameters with organic matrices have a large number of variables that need to be optimized including concentration, solvent composition, spraying temperature and linear flow rate. These variables were also explored for AuNPs and a high degree of flexibility was determined for all of them. Specifically, the following tolerances for ionization were determined: (i) a variety of organic:aqueous solvent mixes are possible, (ii) temperature from 30-75° C. can be utilized, and (iii) spray velocity has a broad range and impacts linear flow rate. In addition, the use of AuNPs has the benefit of only requiring a small number of spraying passes, which significantly reduces sample preparation time, putting this on a clinically feasible timescale. Lastly, greater flexibility with sample storage resulted, including the ability to freeze AuNP-sprayed tissue sections on slides overnight at −20° C. (or store them under vacuum) and repeat imaging runs the following day without additional spraying of AuNPs, with nearly identical data resulting.

### Example 4: Utility of Principal Component Analysis Plots for Optimizing AuNPs for Mass Spectrometry Imaging

### Methods

MSI experiments were performed on zebrafish embryos that were embedded in M1 embedding media. Tissue was sectioned at 10 μm thickness and thaw mounted onto ITO slides at −16° C. AuNPs (Ted Pella; Redding, Calif.) or organic matrix (e.g., DHB) were sprayed onto tissue sections using an HTX Imaging Sprayer. All imaging experiments were performed on Bruker Rapiflex MALDI TOF/TOF in reflectron positive mode at various pixel sizes ranging between 5-200 microns. Sample preparation variables range from: (i) traditional matrix vs. AuNPs, (ii) AuNP size, (iii) spray parameters, and (iv) number of spray passes. Method development investigated the effects of the following: (i) NP concentration, (ii) number of spray passes, (iii) organic:aqueous solvent mixes, (iv) spray temperatures, (v) spray velocity, (vi) NP size and capping agent, and (vii) tissue handling and preparation.

### Results

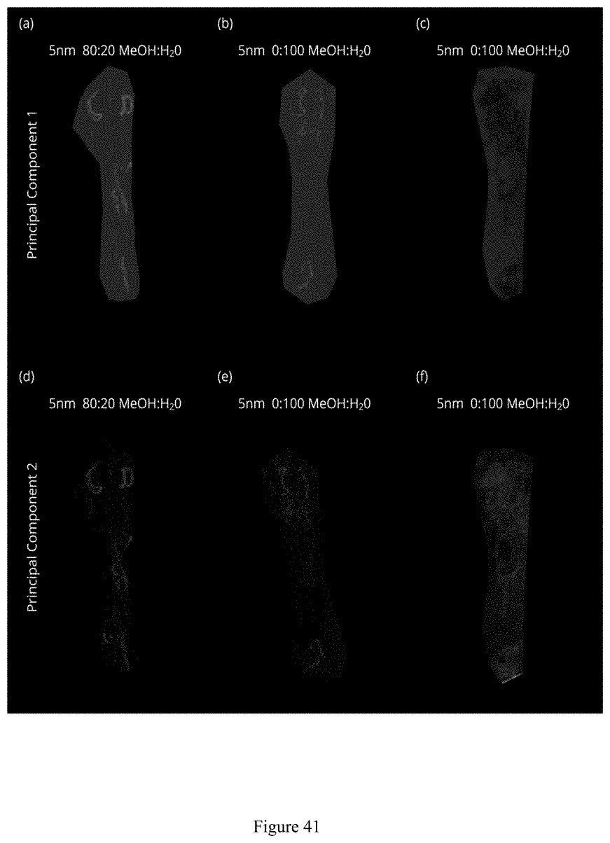

The scores plot from principal components 1 and 2 account for 39% of the total variance of the dataset. An increase in the amount of water in a sample corresponds to an increased amount of delocalization. Qualitatively, the greatest differentiation between spectral profiles of these preparations is captured in the first principal component. The 80:20 MeOH:H₂O normal velocity and 100% water high velocity date have the most similar spectral profiles. Data acquired with 100% water normal velocity is clearly distinguishable from the other preparations. The purple scores with 100% water content suffer increased delocalization while the blue scores have the same water content and overlap with the orange scores. This difference stems from the high velocity represented by the blue scores while a low velocity was used on the purple scores. This insight suggests that there is a large difference in the effects of delocalization on collected data based on the velocity of the instrument and that this difference is quickly detectable using PCA. FIG. 41 demonstrates the quantity of ionization (y-axis) for acetylcholine and GABA/choline using various AuNP preparations. The height of the blue portion represents through the 3^{rd} quartile and the red portion represents outliers. Therefore, the level of ionization between the tannic acid and citrate capped is overshadowed in both cases by the 80:20 MeOH:H₂O.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

What is claimed:

1. A method of imaging neurotransmitters in a biological sample comprising:
a. pneumatically spraying or having sprayed said biological sample with a nanoparticle;
b. introducing said sample to a laser desorption ionization mass spectrometer to collect mass spectral data; and
c. identifying said neurotransmitters in said sample based on said mass spectral data.

2. The method of claim 1, wherein said neurotransmitter is a monoamine.

3. The method of claim 1, wherein said neurotransmitter is an amino acid.

4. The method of claim 1, wherein said neurotransmitter is a monoamine selected from the group consisting of dopamine, octopamine, norepinephrine, epinephrine, serotonin, and histamine.

5. The method of claim 1, wherein neurotransmitter is an amino acid selected from the group consisting of glutamate, gamma-aminobutyric acid (GABA), glycine, and tyramine.

6. The method of claim 1, wherein said neurotransmitter is selected from the group consisting of acetylcholine, adenosine, and nitric oxide.

7. The method of claim 1, wherein said neurotransmitter is present in said biological sample at physiological concentration.

8. The method of claim 1, wherein said biological sample is an organ, tissue, or cell.

9. The method of claim 1, wherein said biological sample is selected from the group consisting of brain tissue, spinal tissue, or peripheral nerve tissue.

10. The method of claim 1, wherein said biological sample is present in neural tissue.

11. The method of claim 1, wherein said biological sample is brain tissue.

12. The method of claim 1, wherein said pneumatic spraying is done with a high volume, low pressure device.

13. The method of claim 1, wherein said spraying is done using a hand spraying device.

14. The method of claim 1, wherein said spraying is done using an airbrush.

15. The method of claim 1, wherein said spraying is done at a temperature of from about 22° C. to about 95° C.

16. The method of claim 1, wherein said spraying is done at a velocity of about 1000 15 mm/minute to about 2000 mm/minute.

17. The method of claim 1, wherein said nanoparticle is a metal nanoparticle selected from the group consisting of gold, silver, and platinum.

18. The method of claim 1, wherein said nanoparticle is gold.

19. The method of claim 1, wherein said nanoparticle is silica.

20. The method of claim 19, wherein said nanoparticle is coated with a metal selected from the group consisting of gold, silver, and platinum.

21. The method of claim 1, wherein said nanoparticle is solid, hollow, a pitted solid, or has at least one open channel therein.

22. The method of claim 1, wherein said nanoparticle is solid.

23. The method of claim 1, wherein said nanoparticle is a solid with an exterior coating.

24. The method of claim 1, wherein said nanoparticle is silica is coated with gold.

25. The method of claim 1, wherein said nanoparticle is substantially in the shape of a sphere, wire, rod, pyramid, double pyramid, diamond, cube, or star.

26. The method of claim 1, wherein in said nanoparticle is substantially in the shape of a sphere.

27. The method of claim 1, wherein a negatively charged surface ligand is adsorbed on the surface of said nanoparticle and said ligand is a carboxylic acid functionality.

28. The method of claim 27, wherein said carboxylic acid functionality is citrate.

29. The method of claim 1, wherein a positively charged surface ligand is adsorbed on the surface of said nanoparticle.

30. The method of claim 29, wherein said positively charged surface ligand is a quaternary amine.

31. The method of claim 1, wherein a neutrally charged surface ligand is adsorbed on the surface of said nanoparticle.

32. The method of claim 31, wherein said neutrally charged surface ligand is selected from the group consisting of tannic acid, dextrin, and dextrans.

33. The method of claim 1, wherein a fluorescent ligand is adsorbed on the surface of said nanoparticle.

34. The method of claim 1, wherein a fluorescent ligand is covalently bound to the surface of said nanoparticle.

35. The method of claim 1, wherein said nanoparticle ranges in size from about 1 nm to about 50 nm in their longest dimension.

36. The method of claim 1, wherein said nanoparticle is coated with both a metal and a fluorescent ligand.

37. The method of claim 1, wherein mass spectral data are obtained for more than one neurotransmitter in said biological sample.

38. A method of imaging metabolites in a biological sample comprising:
a. pneumatically spraying or having sprayed said biological sample with a nanoparticle;
b. introducing said sample to a laser desorption ionization mass spectrometer to collect mass spectral data; and
c. identifying said metabolite in said sample based on said mass spectral data.

39. The method of claim 37, wherein said metabolite is selected from the group consisting of glucose, pyruvate, NAD, NADH, ATP, ADP, FAD, and FADH.

40. A mass spectrometer sample prepared by pneumatically spraying or having sprayed said biological sample with a nanoparticle.

41. A biological sample prepared by pneumatically spraying or having sprayed said biological sample with a nanoparticle for analysis in a mass spectrometer.
